(19) United States
(12) Patent Application Publication
(10) Pub. No.: US 2025/0367386 A1
(43) Pub. Date: Dec. 4, 2025

(54) MEDICAL INJECTIONS AND RELATED DEVICES AND METHODS

(71) Applicant: Gilead Sciences, Inc., Foster City, CA (US)

(72) Inventors: Patricia Das, Hertfordshire (GB); Robert Glover, Hertfordshire (GB); Walter Goodwin, San Carlos, CA (US); Woo Suk Kim, Hertfordshire (GB); James Paul Oberhauser, Saratoga, CA (US); Donald Steel, Hertfordshire (GB); Adam Toner, Harpenden (GB); Alasdair Young, Foster City, CA (US)

(21) Appl. No.: 19/206,160

(22) Filed: May 13, 2025

Related U.S. Application Data
(60) Provisional application No. 63/647,273, filed on May 14, 2024.

(51) Int. Cl. A61M 5/315; A61K 9/00; A61K 31/4439; A61K 47/10; A61M 5/20

(52) U.S. Cl. CPC A61M 5/3157; A61K 9/0019; A61K 31/4439; A61K 47/10; A61M 5/20; A61M 2205/332; A61M 2205/3368; A61M 2205/581; A61M 2205/582

(57) ABSTRACT

A medicament delivery device includes a housing, a container disposed within the housing and configured to contain medicament, a plunger slidably disposed within the container, a plunger rod configured to push the plunger through the container to dispense the medicament when the container contains medicament, and a gas canister disposed within the plunger rod and containing a compressed gas, wherein the gas canister is configured to release the compressed gas to drive the plunger rod to dispense the medicament out of a distal end of the container.

### CROSS REFERENCE TO RELATED APPLICATIONS

This application claims priority to U.S. Provisional Application No. 63/647,273, filed May 14, 2024, the entire content of which application is hereby incorporated by reference in its entirety.

### TECHNICAL FIELD

This disclosure relates to medical injections and related devices and methods.

### BACKGROUND

An injection typically refers to the act of administering a liquid (e.g., a drug) into a patient's body tissue. Injecting medicament into a patient can allow the medicament to be absorbed relatively rapidly.

### SUMMARY

The present systems, devices, and methods can sense a dispensing movement of a plunger rod within an autoinjector. For example, an internal component, such as an audible clicker, of the autoinjector can produce signals that can be sensed by a sensing assembly to track the dispensing movement of the plunger rod. The signals can include audible clicks, vibrations, and the like. The devices can include a sensing assembly that can determine that the plunger rod has completed the dispensing movement after receiving a predetermined number of signals. The sensing assembly can also track the dispensing movement of the plunger rod. Tracking the dispensing movement of the plunger rod can be advantageous because the dispensing movement of the plunger rod corresponds to the amount of medicament delivered to the patient. If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth and/or whether the insertion depth is maintained during delivery of the medicament. For example, a force sensor can determine whether an insertion force is greater than a threshold force to insert the needle a sufficient distance into the patient. Determining that the needle is sufficiently inserted into the patient can be advantageous because if the needle is not sufficiently inserted into the patient, the patient may not receive the medicament properly.

The present disclosure relates to a lenacapavir composition for use in the prevention or treatment of HIV, where the lenacapavir is administered by an autoinjector according to this disclosure. The present disclosure also relates to a use of lenacapavir for the manufacture of a medicament for the prevention or treatment of HIV, where the lenacapavir is administered by an autoinjector according to the disclosure.

In a first aspect of the invention, the present disclosure encompasses an autoinjector including: a housing; a container disposed within the housing and configured to contain medicament; a plunger slidably disposed within the container; a plunger rod configured to push the plunger through the container to dispense the medicament when the container contains medicament; and an audible clicker contacting the plunger rod. A dispensing movement of the plunger rod may deflect the audible clicker causing the audible clicker to produce one or more audible clicks. The autoinjector may further include: a sensor configured to detect the one or more audible clicks of the audible clicker as the plunger rod moves.

In a second aspect of the invention, the present disclosure encompasses an autoinjector including: a housing; a container disposed within the housing and configured to contain medicament; a plunger slidably disposed within the container; a plunger rod configured to push the plunger through the container to dispense the medicament when the container contains medicament; and a mechanism configured so that a dispensing movement of the plunger rod causes the mechanism to generate a sound. The autoinjector may further include: a sensor configured to detect sound generated by the mechanism.

According to the first or second aspects of the invention, the audible clicker may be configured to produce a plurality of audible clicks during the dispensing movement of the plunger rod. Optionally, the audible clicker may include a ring surrounding the plunger rod. The ring may include a deflectable protrusion that is configured to contact the plunger rod and produces the one or more audible clicks. Further optionally, the plunger may include a ridged surface that is configured to contact the deflectable protrusion of the audible clicker.

In a third aspect of the invention, the present disclosure encompasses an autoinjector including: a housing; a container disposed within the housing and configured to contain medicament; a plunger slidably disposed within the container; a plunger rod configured to push the plunger through the container to dispense the medicament when the container contains medicament; a deflectable protrusion contacting the plunger rod, wherein a dispensing movement of the plunger rod deflects the deflectable protrusion; and a sensor configured to detect one or more deflections of the deflectable protrusion as the plunger rod moves.

In a fourth aspect of the invention, the present disclosure encompasses an autoinjector including: a housing; a container disposed within the housing and configured to contain medicament; a plunger slidably disposed within the container; a plunger rod configured to push the plunger through the container to dispense the medicament when the container contains medicament; a mechanism configured so that a dispensing movement of the plunger rod causes the mechanism to vibrate; and a sensor configured to detect vibration generated by the mechanism.

In a fifth aspect of the invention, the present disclosure encompasses a system including: an autoinjector (e.g., any described herein, such as the first, second, third or fourth aspects of the invention); and a processor configured to process data generated by a sensor to track the dispensing movement of the plunger rod.

In a sixth aspect of the invention, the present disclosure encompasses an autoinjector including: a housing; a needle arranged at a distal end of the housing; a force sensor configured to detect that an insertion force is greater than a threshold force; and a processor configured to use the detected insertion force to determine when the insertion force is greater than a threshold insertion force, and thus determine that an insertion distance of the needle into a user is greater than a threshold insertion distance.

In a seventh aspect of the invention, the present disclosure encompasses a system including: an autoinjector including a housing; a needle arranged at a distal end of the housing; and a force sensor configured to detect that an insertion force is greater than a threshold force; and a processor external to the autoinjector. The processor may be configured to use the detected insertion force to determine when the insertion force is greater than a threshold insertion force, and thus determine that an insertion distance of the needle into a user is greater than a threshold insertion distance.

According to any of the first to seventh aspects of the invention, where the autoinjector comprises a container configured to contain medicament, the container may contain the medicament. Optionally, the medicament comprises lenacapavir or a pharmaceutically accepted salt thereof. Additionally or alternatively, where the autoinjector comprises a plunger rod, a container and a plunger, the autoinjector may further comprise a gas canister assembly configured to release pressurized gas which, when released, provides a force acting on the plunger rod to push the plunger through the container. The one or more audible clicks, the one or more deflections, the vibration or the sound may be configured to indicate dose progression. The one or more audible clicks, the one or more deflections, the vibration or the sound may be configured to be detected for tracking the dispensing movement. The one or more audible clicks, the one or more deflections, the vibration or the sound may be configured to indicate completion of the dispensing movement of the plunger rod.

In an eighth aspect of the invention, the present disclosure encompasses a method of detecting a dispensing movement of a plunger rod within an autoinjector. The method includes detecting, using a sensor, one or more audible clicks of an audible clicker as the plunger rod moves during the dispensing movement. The dispensing movement of the plunger rod may deflect the audible clicker causing the audible clicker to produce the one or more audible clicks.

In a ninth aspect of the invention, the present disclosure encompasses a method of detecting a dispensing movement of a plunger rod within an autoinjector. The method includes detecting a sound generated due to movement of the plunger rod during the dispensing movement.

In a tenth aspect of the invention, the present disclosure encompasses a method of detecting a dispensing movement of a plunger rod within an autoinjector including a plunger rod and a protrusion contacting the plunger rod. The method includes detecting, using a sensor, one or more deflections of the protrusion as the plunger rod moves during the dispensing movement, wherein the dispensing movement of the plunger rod deflects the protrusion.

In an eleventh aspect of the invention, the present disclosure encompasses a method of detecting a dispensing movement of a plunger rod within an autoinjector including a component. The method includes detecting a deflection of the component due to movement of the plunger rod during the dispensing movement.

According to a twelfth aspect of the invention, also provided is a composition comprising lenacapavir or a pharmaceutically accepted salt thereof for use in the prevention or treatment of HIV, where the composition is administered via an autoinjector (e.g., any described herein, such as those of the first, second, third, fourth, fifth, sixth or seventh aspects of the invention). The administration may be subcutaneous or intramuscular.

According to a thirteenth aspect of the invention, also provided is the use of lenacapavir or a pharmaceutically accepted salt thereof for the manufacture of a medicament for the prevention or treatment of HIV, wherein the prevention or treatment comprises administering the medicament via an autoinjector (e.g., any described herein, such as those of the (e.g., any described herein, such as those of the first, second, third, fourth, fifth, sixth or seventh aspects of the invention). The administration may be subcutaneous or intramuscular.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIGS. 1A and 1B illustrate an example of an autoinjector.

FIGS. 2A-E illustrate examples of an audible clicker and a plunger rod, respectively.

FIGS. 3A and 3B illustrate examples of a plunger rod and an example sensing output, respectively.

FIG. 4 illustrates an example of a PCB sub-assembly.

FIGS. 5A-F illustrate an example of a sensing assembly for an autoinjector.

FIG. 6 illustrates a sensing assembly in a proximal portion of an autoinjector.

FIG. 7 illustrates an example method of using an autoinjector.

FIGS. 8A-F example internal components of an autoinjector and firing of the autoinjector.

Like reference numbers and designations in the various drawings indicate like elements.

### DETAILED DESCRIPTION

The present systems, devices, and methods can sense a dispensing movement of a plunger rod within an autoinjector. For example, an internal component, such as an audible clicker, of the autoinjector can produce signals that can be sensed by a sensing assembly to track the dispensing movement of the plunger rod. The signals can include audible clicks, vibrations, and the like. The devices can include a sensing assembly that can determine that the plunger rod has completed the dispensing movement after receiving a predetermined number of signals. The sensing assembly can also track the dispensing movement of the plunger rod. Tracking the dispensing movement of the plunger rod can be advantageous because the dispensing movement of the plunger rod corresponds to the amount of medicament delivered to the patient. If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth. For example, a force sensor can determine whether an insertion force is greater than a threshold force to insert the needle a sufficient distance into the patient. Determining that the needle is sufficiently inserted into the patient can be advantageous because if the needle is not sufficiently inserted into the patient, the patient may not receive the medicament properly.

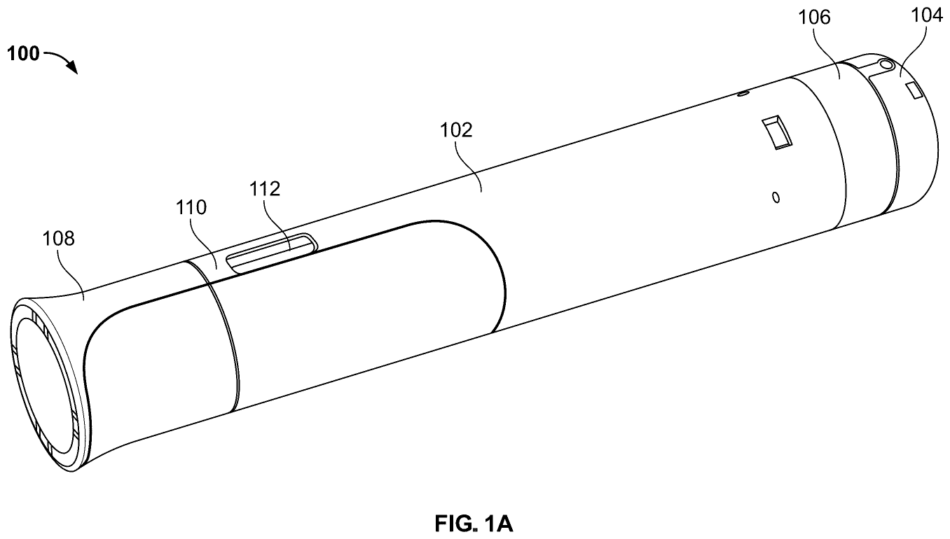

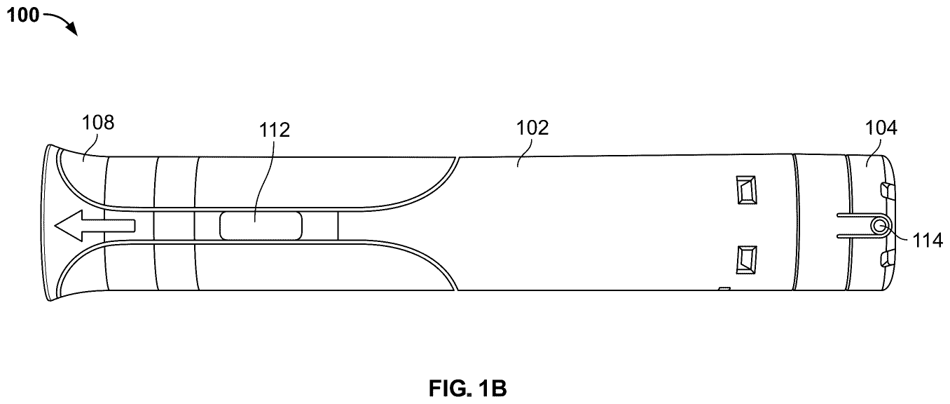

FIG. 1A illustrates an autoinjector 100 that can sense a dispensing movement of a plunger rod within the autoinjector 100 and sense whether a needle of the autoinjector 100 is inserted into a patient to a sufficient depth. Alternatively or in addition, the autoinjector 100 can sense whether an insertion depth of a needle is maintained during dispensing or delivery. The autoinjector 100 includes a housing 102, a sensing assembly 104 on a proximal end 106 of the housing 102, and a cap 108 attached to a distal end 110 of the housing 102. The cap 108 covers a needle assembly configured to be inserted into the user during injection. The user removes the cap 108 prior to use of the autoinjector 100. The sensing assembly 104 may be located elsewhere on the autoinjector. For example, the sensing assembly 104 can be located somewhere between the proximal end 106 and the distal end 110 of the housing 102. The sensing assembly 104 can sense a dispensing movement of a plunger rod within the autoinjector 100, e.g., to determine that the plunger rod has completed the dispensing movement, as discussed below. The housing 102 may include flanges configured to accommodate the user's fingers.

The housing 102 can include a label that provides information about the autoinjector 100. For example, the label can include medicament information, such as the type of medicament, the size of the dose, and the delivery time of the dose. Optionally, the housing 102 does not include a label.

The housing 102 also includes an optional window 112, through which a user can see medicament contained within the autoinjector 100, e.g., within a container of the autoinjector 100 (see discussion below). The window 112 may help a user determine whether the autoinjector 100 has been used. Before use of the autoinjector 100, the user can see through the window 112 to determine whether there is medicament within the autoinjector 100, for example, to determine that the autoinjector has not been used. During use of the autoinjector 100, the user may look through the window 112 to determine whether the volume of medicament in the autoinjector 100 is decreasing. After use of the autoinjector 100, the user may look through the window 112 to determine that there is no medicament in the autoinjector 100, for example, to determine that the autoinjector 100 has been used.

FIG. 1B illustrates an example schematic of an autoinjector 100 including a cap 108, a window 112, a housing 102, a sensing assembly 104, and an optional button 114 to power up or wake up the sensing assembly 104.

Different injection sites, patient age and patient body mass may affect the recommended needle length, and higher viscosity drugs will require a larger diameter needle to prevent the injection force becoming too high for the device. Advantageously, selecting needle gauge based on viscosity of drug being administered can ensure that the full dose of drug is administered without undue strain. Typically, injection force is less than 40 Newtons through needle gauge selection. Preferably, injection force is less than 20 Newtons through needle gauge selection.

Needle gauges disclosed herein are provided in Birmingham Wire Gauge (also known as: Birmingham Gauge or Stubs Iron Wire Gauge), abbreviated as “gauge” or G. In accordance with ISO standard ISO 9626:2016, needle wall thickness designations include Regular Wall, Thin Wall, Extra Thin Wall, and Ultra Thin Wall. Regular Wall thickness is abbreviated to RW. Thin Wall thickness is abbreviated to TW. Extra Thin Wall thickness is abbreviated to ETW. Ultra Thin Wall is abbreviated to UTW. Alternatively, needle wall thickness may be Special Thin Wall; Special Thin Wall thickness is abbreviated as STW. Viscosity is provided in centipoise (cP), where one centipoise is equivalent to one millipascal-second.

The autoinjector 100 may be used for subcutaneous injections, which are directed into fat tissue between the skin and the muscle of the patient. Subcutaneous injections can involve shorter and/or wider needles than intramuscular injections, which are directed into the muscle of a patient. As an example, when autoinjector 100 is used for subcutaneous injections, the needle may be 20 gauge and 1.5 inches long. As another example, the needle may be 27 gauge and 0.5 inches long. The needle can have a variety of gauges (e.g., 23-25 gauge, less than 25 gauge, 18-25 gauge, etc.) for subcutaneous injections. Insertion depth for a subcutaneous injection may be 4-8 mm. For subcutaneous injection, needle length may be 8-13 mm. For delivery of 2.25-3 mL dose of liquid (medicament) with a viscosity of up to 5 cP using a needle with a needle length 8-13 mm, needle gauge may be 29 G RW or TW, or 27 G RW. For delivery of 2.25-3 mL dose of liquid (medicament) with a viscosity of up to 30 cP using a needle with a needle length 8-13 mm, needle gauge may be 27 G TW or 25 G RW. For delivery of 2.25-3 mL dose of liquid (medicament) with a viscosity of up to 50 cP using a needle with a needle length 8-13 mm, needle gauge may be 25 G TW. For delivery of 2.25-3 mL dose of liquid (medicament) with a viscosity of up to 160 cP using a needle with a needle length 8-13 mm, needle gauge may be 25 G STW or 23 G RW. For delivery of 2.25-3 mL dose of liquid (medicament) with a viscosity of up to 300 cP using a needle with a needle length 8-13 mm, needle gauge may be 22 G ETW. For delivery of 2.25-3 mL dose of liquid (medicament) with a viscosity of up to 600 cP using a needle with a needle length 8-13 mm, needle gauge may be 18 G ETW or 18 G UTW. For delivery of 1 mL dose of liquid (medicament) with a viscosity of up to 10 cP using a needle with a needle length 8-13 mm, needle gauge may be 29 G RW or TW, or 27 G RW. For delivery of 1 mL dose of liquid (medicament) with a viscosity of up to 30 cP using a needle with a needle length 8-13 mm, needle gauge may be 27 G TW or 25 G RW. For delivery of 1 mL dose of liquid (medicament) with a viscosity of up to 80 cP using a needle with a needle length 8-13 mm, needle gauge may be 25 G TW. For delivery of 1 mL dose of liquid (medicament) with a viscosity of up to 350 cP using a needle with a needle length 8-13 mm, needle gauge may be 25 G STW or 23 G RW. For delivery of 1 mL dose of liquid (medicament) with a viscosity of up to 600 cP using a needle with a needle length 8-13 mm, needle gauge may be 18-22 G ETW.

The autoinjector 100 may be used for intramuscular injections. The needle can also have a variety of lengths (e.g., at least 1.5 inches, 1.5-2 inches, 1-2 inches, 0.75-2.25 inches, etc.) for intramuscular injections. A target insertion length of the needle can be from 3 millimeters to 5 millimeters (e.g., 3 millimeters, 3.5 millimeters, 4 millimeters, 4.5 millimeters, 5 millimeters) for intramuscular injections. The needle can have a variety of gauges (e.g., 20-21 gauge, less than 25 gauge, 18-25 gauge, etc.) for intramuscular injections. Alternatively, insertion depth for intramuscular injection may be 25-50 mm. For intramuscular injection, needle length may be 1-1.5 inches (25.4-38.1 mm). For delivery of 2.25-3 mL dose of liquid (medicament) with a viscosity of up to 1 cP using a needle with a needle length 25.4-38.1 mm, needle gauge may be 29 G RW or TW, or 27 G RW. For delivery of 2.25-3 mL dose of liquid (medicament) with a viscosity of up to 5 cP using a needle with a needle length 25.4-38.1 mm, needle gauge may be 27 G TW or 25 G RW. For delivery of 2.25-3 mL dose of liquid (medicament) with a viscosity of up to 10 cP using a needle with a needle length 25.4-38.1 mm, needle gauge may be 25 G TW. For delivery of 2.25-3 mL dose of liquid (medicament) with a viscosity of up to 40 cP using a needle with a needle length 25.4-38.1 mm, needle gauge may be 25 G STW or 23 G RW. For delivery of 2.25-3 mL dose of liquid (medicament) with a viscosity of up to 200 cP using a needle with a needle length 25.4-38.1 mm, needle gauge may be 22 G ETW. For delivery of 2.25-3 mL dose of liquid (medicament) with a viscosity of up to 600 cP using a needle with a needle length 25.4-38.1 mm, needle gauge may be 18 G ETW or 18 G UTW. For delivery of 1 mL dose of liquid (medicament) with a viscosity of up to 5 cP using a needle with a needle length 25.4-38.1 mm, needle gauge may be 29 G RW or TW, or 27 G RW. For delivery of 1 mL dose of liquid (medicament) with a viscosity of up to 10 cP using a needle with a needle length 25.4-38.1 mm, needle gauge may be 27 G TW or 25 G RW. For delivery of 1 mL dose of liquid (medicament) with a viscosity of up to 30 cP using a needle with a needle length 25.4-38.1 mm, needle gauge may be 25 G TW.

For delivery of 1 mL dose of liquid (medicament) with a viscosity of up to 50 cP using a needle with a needle length 25.4-38.1 mm, needle gauge may be 25 G STW or 23 G RW. For delivery of 1 mL dose of liquid (medicament) with a viscosity of greater than 50 cP using a needle with a needle length 25.4-38.1 mm, needle gauge may be 18-22 G ETW.

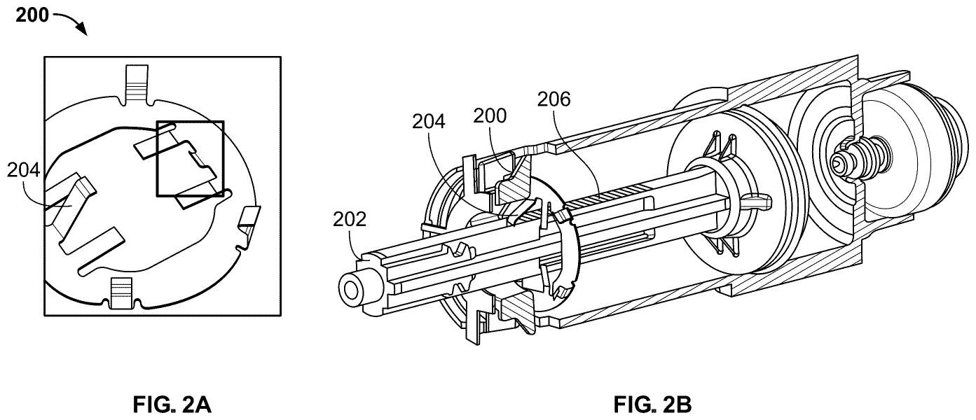

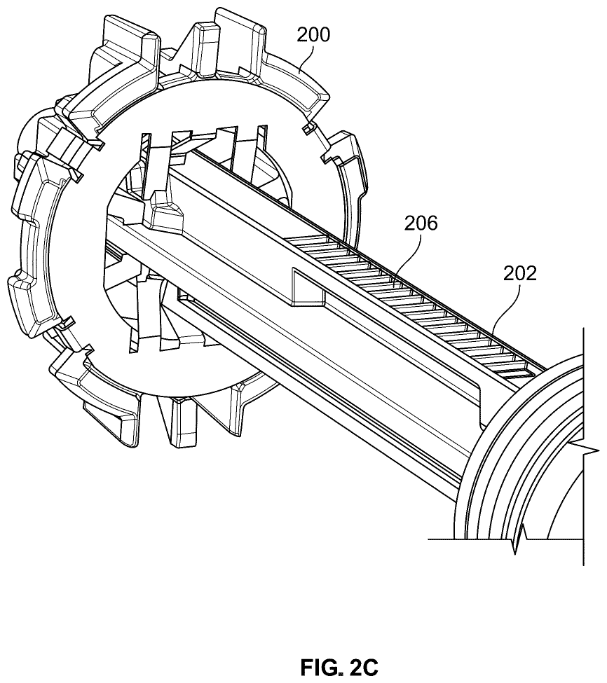

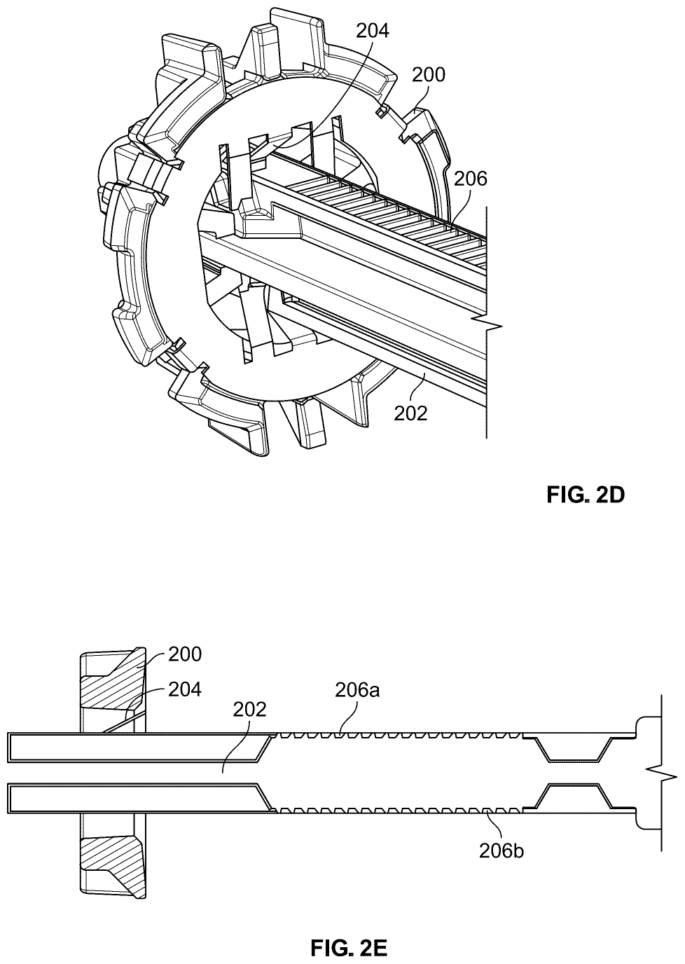

FIGS. 2A-E illustrate examples of an audible clicker 200 and a plunger rod 202 that can serve as internal components of an autoinjector (e.g., an autoinjector similar to the autoinjector 100 of FIGS. 1A-B). For example, the plunger rod 202 can push a plunger through a medicament container to dispense medicament from the medicament container (see discussion below). The plunger rod 202 contacts the audible clicker 200 as the plunger rod 202 moves through a dispensing movement. The audible clicker 200 produces sounds, vibrations, or the like that can be detected (e.g., by a sensing assembly, see below) to track the dispensing movement of the plunger rod 202. For example, the audible clicker 200 can be in the form of a clicker ring having a deflectable protrusion 204 that contacts the plunger rod 202. The dispensing movement of the plunger rod 202 deflects the deflectable protrusion 204 of the audible clicker 200 to cause the audible clicker 200 to produce one or more audible clicks, vibrations, or the like. A surface 206 of the plunger rod 202 can be configured to deflect the deflectable protrusion 204 (see discussion below).

The audible clicker 200 may include multiple deflectable protrusions 204. Optionally, when the audible clicker 200 includes multiple deflectable protrusions 204, the plunger rod 202 can include multiple surfaces configured to deflect the deflectable protrusions 204. For example, and without limitation, as seen in FIG. 2E, the plunger rod 202 can include multiple surfaces 206a/b configured to deflect the deflectable protrusions 204, in which each surface 206a/b can in turn include a plurality of ridges, bumps, projections, or other non-continuous structures along the surface 206a/b to provide a ridged surface or other non-continuous surface (see discussion below). Alternatively, the audible clicker 200 includes a single deflectable protrusion 204. Optionally, when the audible clicker 200 includes a single deflectable protrusion 204, the plunger rod 202 can include a single surface configured to deflect the deflectable protrusion 204. Any number of deflectable protrusions can be employed to produce one or more clicks (e.g., a single deflectable protrusion or multiple deflectable protrusions). In one non-limiting example, a single deflectable protrusion can be employed to reduce the possibility of interference between multiple audible clicks. In another non-limiting example, multiple deflectable protrusions can be employed to operate in sync to provide a more distinct click sound, in which interference can be minimized, e.g., by control of tolerancing and/or alignment.

The audible clicker 200 can be a portion of the autoinjector housing. For example, the autoinjector housing can include a deflectable protrusion that contacts the plunger rod 202. The plunger rod 202 can include one or more deflectable protrusions, and the housing (or another internal component of the autoinjector) may include a surface configured to deflect the one or more deflectable protrusions.

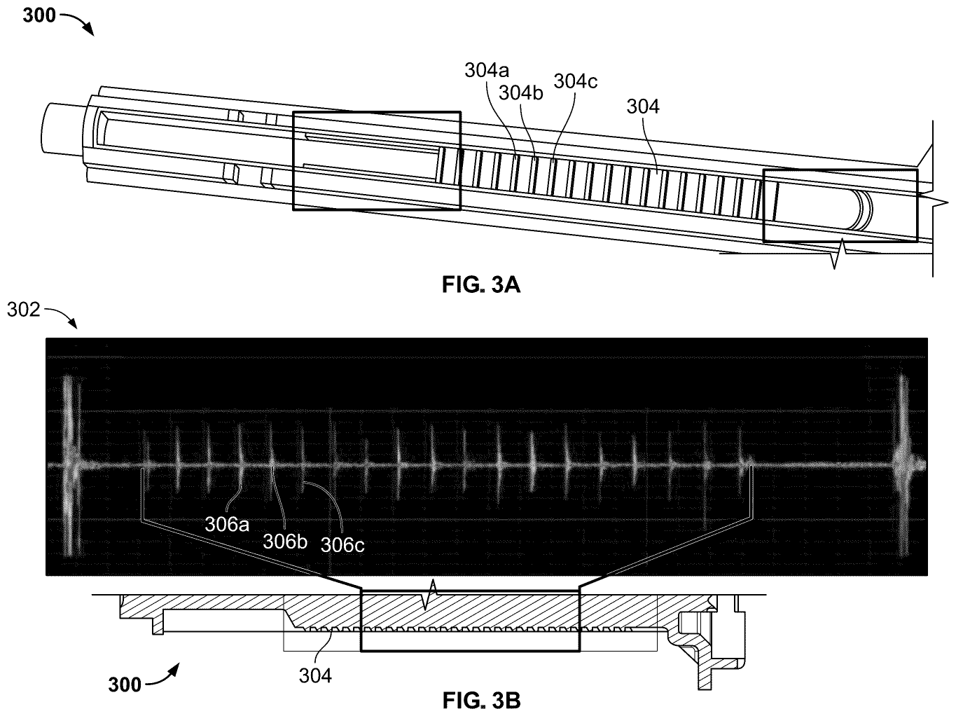

FIGS. 3A and 3B illustrate examples of a plunger rod 300 and an example sensing output signal 302, respectively. The plunger rod 300 includes a ridged surface 304 configured to contact a deflectable protrusion (e.g., of an audible clicker, of a housing, or another appropriate component of the autoinjector). The ridged surface 304 can include any appropriate number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of ridges 304a, 304b, 304c. Each ridge 304a, 304b, 304c of the ridged surface 304 can deflect the deflectable protrusion to cause the deflectable protrusion to produce a signal (e.g., an audible click, a vibration, etc.) that can be sensed by a sensing assembly, as discussed below. For example, referring to FIG. 3B, the example sensing output 302 includes multiple signals 306a, 306b, 306c that correspond to ridges of the ridged surface 304. Each of the signals 306a, 306b, 306c represents a signal received by the sensing assembly. Because each signal 306a, 306b, 306c corresponds with one of the ridges 304a, 304b, 304c of the ridged surface 304, the signals 306a, 306b, 306c can be used to track the dispensing movement of the plunger rod 300. For example, a sensing assembly can use the received signals to determine that the plunger rod 300 has completed the dispensing movement after a predetermined number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of signals. The ridged surface may be any non-continuous surface configured to provide a plurality of non-continuous structures (e.g., bumps, projections, ribs, and the like upon that surface).

The ridged surface 304 may be positioned on the plunger rod 300 such that a first signal occurs when the plunger rod 300 contacts a stopper within a medicament container (e.g., the first ridge of the ridged surface 304 is positioned so that the first ridge will contact the deflectable protrusion when the plunger rod 300 contacts the stopper). The ridged surface 304 may be positioned on the plunger rod 300 such that a last signal occurs when the plunger rod 300 completes the dispensing movement (e.g., the last ridge of the ridged surface 304 is positioned so that the last ridge will contact the deflectable protrusion when the plunger rod 300 completes the dispensing movement). Alternatively, the ridged surface 304 may be positioned on the plunger rod 300 such that the last signal occurs before the plunger rod 300 completes the dispensing movement (e.g., the last ridge of the ridged surface 304 is positioned so that the last ridge will contact the deflectable protrusion before the plunger rod 300 completes the dispensing movement).

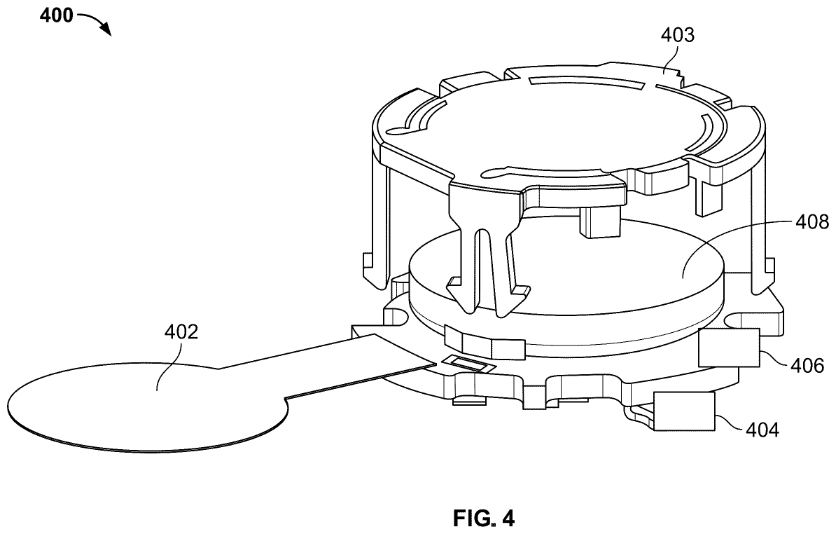

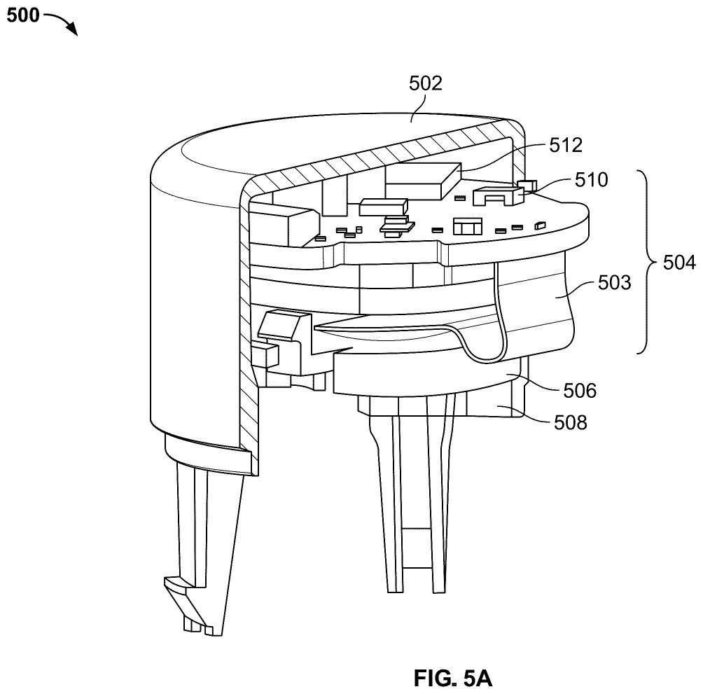

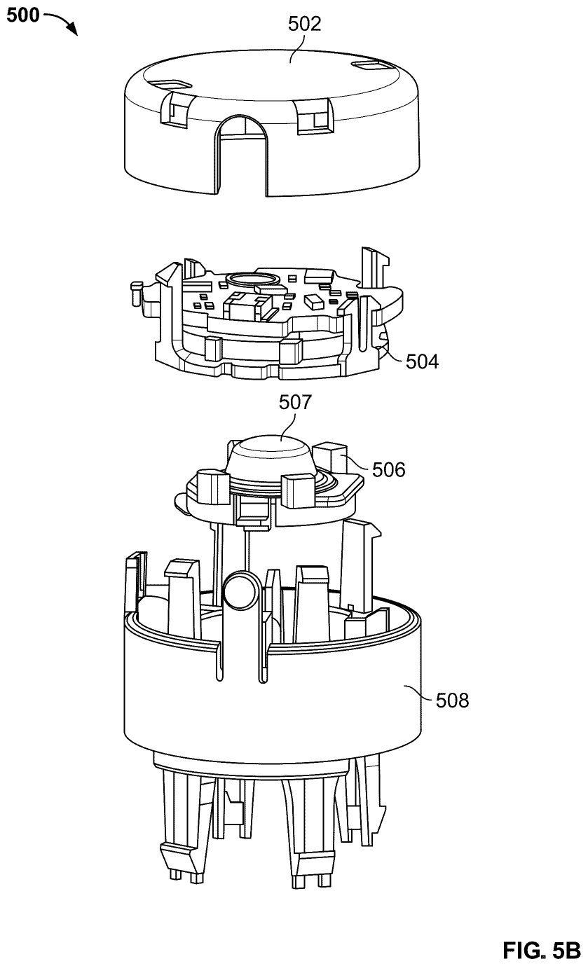

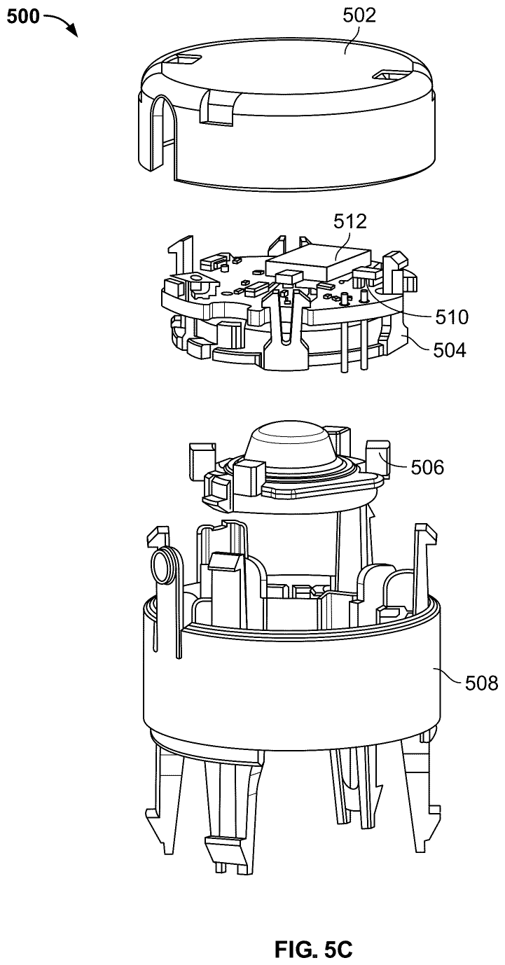

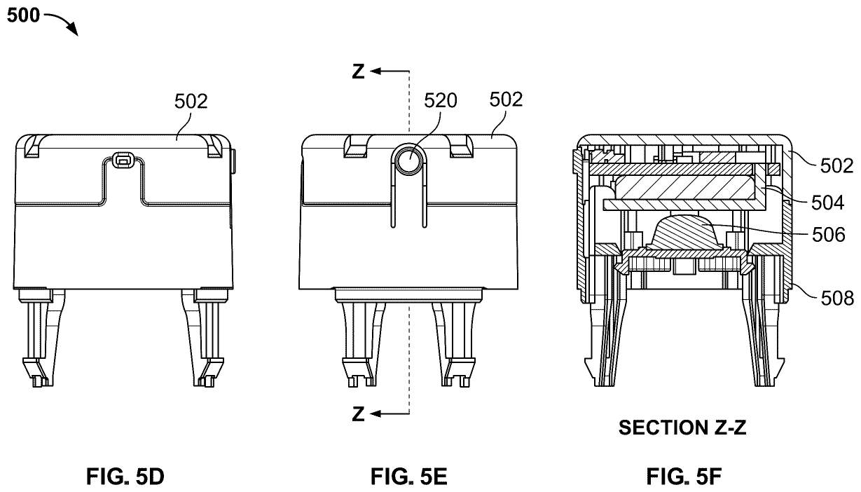

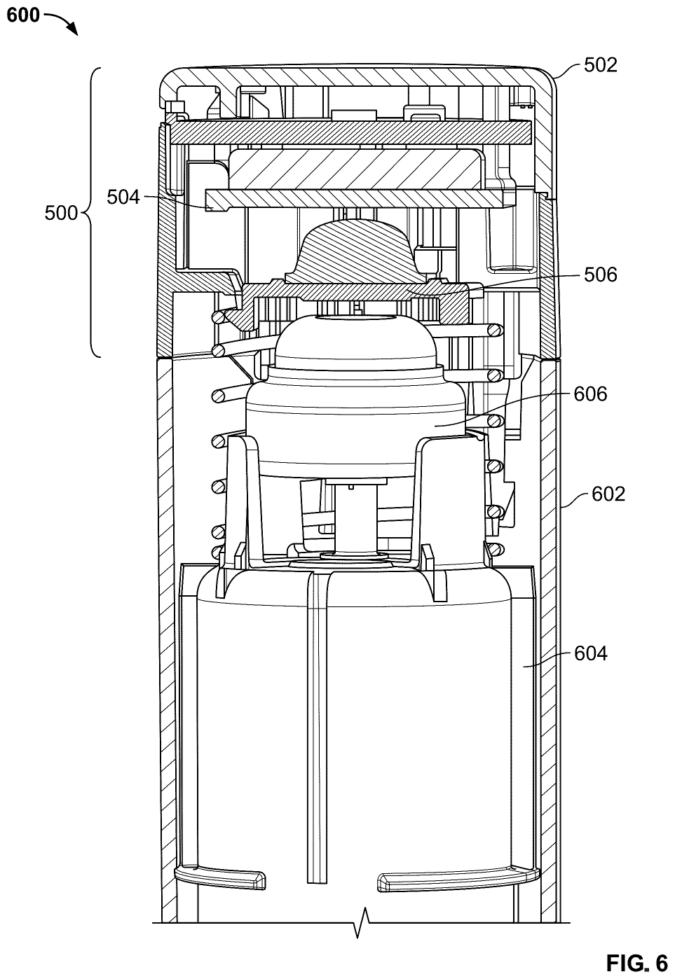

FIG. 4 illustrates a PCB sub-assembly 400 including a force sensor 402, a vibration sensor 404, a wireless transfer protocol module 406, and a battery 408. The force sensor 402 can be disposed on a flexible tab, which in turn can fold over and be mounted on a plate 403 for assembly. The placement and orientation of components within the PCB sub-assembly 400 may be configured for optimal sensing and/or signal transmission. Another non-limiting PCB sub-assembly 504 is illustrated in FIGS. 5A-F, which includes PCB sub-assembly 400 having additional outer housing components. Examples of a force sensor include a force sensitive resistor, a load cell, a strain gauge, a force sense capacitor. The force sensor 402 can measure an insertion force used to insert the needle of the autoinjector a sufficient distance into the patient. For example, an internal component of an autoinjector can contact the force sensor 402 with a force corresponding to an insertion force by the user, as discussed further below with reference to FIG. 6. Examples of a vibration sensor include an accelerometer, a microphone (e.g., a contact microphone or an air microphone), a displacement sensor, a velocity sensor. The vibration sensor may be a sound sensor. The vibration sensor may be a mechanical vibration sensor. The vibration sensor can sense a dispensing movement of a plunger rod that contacts an audible clicker, as discussed above. For example, the vibration sensor 404 can sense the vibrations, sounds, etc. caused by the deflection of the deflectable protrusion. The placement of the vibration sensor 404 in the PCB sub-assembly 400 may be configured to sensing of vibrations, sounds, etc.

The wireless transfer protocol module 406 can receive signals from the force sensor 402 and the vibration sensor 404, and the wireless transfer protocol module 406 can communicate the signals to a processor (e.g., internal to the autoinjector or external to the autoinjector, such as in a mobile device) for further processing. For example, the processor can use the measured insertion force to determine whether the insertion force is greater than a predetermined insertion force threshold. If the insertion force is greater than the predetermined insertion force threshold (e.g., 5 Newtons (N), 10 N, 15 N, 10-20 N, 5-25 N, 12-30 N), the processor can determine that the needle has been inserted an appropriate depth (e.g., 4 mm, 4.5 mm, 5 mm, 3-5 mm) into the patient. The processor can also use the measured vibrations from the vibration sensor to track the dispensing movement of the plunger rod, as discussed above. The processor may be included in the PCB sub-assembly.

The sensing assembly is intended to include various forms of digital computers, such as printed circuit boards (PCB), processors, digital circuitry, or otherwise parts of a system for determining dose progression of an autoinjector. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The sensing assembly can include a processor, a memory, a storage device, and an input/output device (for example, sensors). Each of the components is interconnected using a system bus. The sensing assembly is capable of processing instructions for execution within the sensing assembly. The sensing assembly may be designed using any of a number of architectures. For example, the sensing assembly can include a CISC (Complex Instruction Set Computers) processor, a RISC (Reduced Instruction Set Computer) processor, or a MISC (Minimal Instruction Set Computer) processor.

In one implementation, the processor is a single-threaded processor. In another implementation, the processor is a multi-threaded processor. The processor is capable of processing instructions stored in the memory or on the storage device to display graphical information for a user interface on an input/output device.

The memory stores information within the sensing assembly. In one implementation, the memory is a computer-readable medium. In one implementation, the memory is a volatile memory unit. In another implementation, the memory is a non-volatile memory unit.

The storage device is capable of providing mass storage for the sensing assembly. In one implementation, the storage device is a computer-readable medium. In various different implementations, the storage device may be a floppy disk device, a hard disk device, an optical disk device, or a tape device.

The features described can be implemented in digital electronic circuitry, or in computer hardware, firmware, software, or in combinations of them. The features can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

FIGS. 5A-F illustrate an example of a sensing assembly 500 for an autoinjector. FIGS. 5A, D-F illustrate the sensing assembly 500 in an assembled state, and FIGS. 5B-C illustrate the sensing assembly 500 in a disassembled state. The sensing assembly 500 includes a rear cap 502, a PCB sub-assembly 504, a floating probe assembly 506, and a rear cap retainer 508. The PCB sub-assembly 504 can be similar to the PCB sub-assembly 400 of FIG. 4 and can include a force sensor 503, a vibration sensor 510, and a wireless transfer protocol module 512. The rear cap 502 can form a part of a housing for the sensing assembly 500, retaining the PCB sub-assembly 504 and the floating probe assembly 506. The rear cap 502 connects to the rear cap retainer 508, which can be connected to a housing of an autoinjector (e.g., similar to the housing 102 of FIGS. 1A-1B). The rear cap 502 can be attached and unattached from the rear cap retainer 508 to connect the PCB sub-assembly 504 and the floating probe assembly 506 to the autoinjector and remove the PCB sub-assembly 504 and the floating probe assembly 506 from the autoinjector. Optionally, the rear cap retainer 508 can be attached and unattached from the autoinjector to connect the PCB sub-assembly 504 and the floating probe assembly 506 to the autoinjector and remove the PCB sub-assembly 504 and the floating probe assembly 506 from the autoinjector.

The floating probe assembly 506 acts as a contact between internal components of the autoinjector and the force sensor of the PCB sub-assembly 504. Adding a floating probe assembly 506 can be advantageous, e.g., to spread the load between an internal component of the autoinjector and the force sensor in the PCB sub-assembly 504. Spreading the load between the internal component and the force sensor can reduce variability in the force sensor measurements. The PCB sub-assembly 504 may contact the floating probe assembly 506 by way of a domed component 507 of the floating probe assembly 506, with reference to FIG. 5A. The PCB sub-assembly 504 may contact the floating probe assembly 506 directly by way of any component or structure of the PCB sub-assembly 504. Optionally, the sensing assembly 500 can also include a temperature sensor (e.g., a thermistor). Optionally, the sensing assembly 500 can power up or wake up upon activation of a button 520. Optionally, the sensing assembly 500 can power up or wake up upon activation of a component with the PCB sub-assembly 504 (e.g., a force sensor within the PCB sub-assembly 504). Optionally, the sensing assembly 500 can power up or wake up by remote activation (e.g., from an app and/or website on the mobile device, as discussed below).

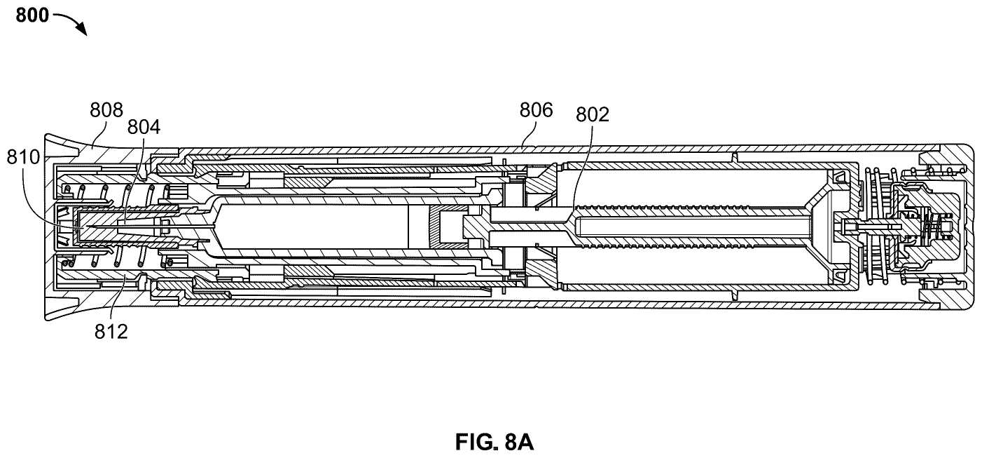

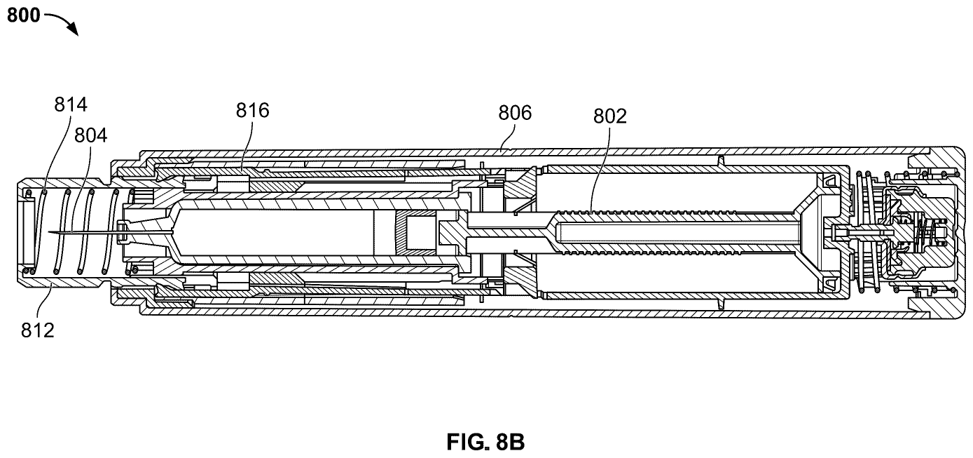

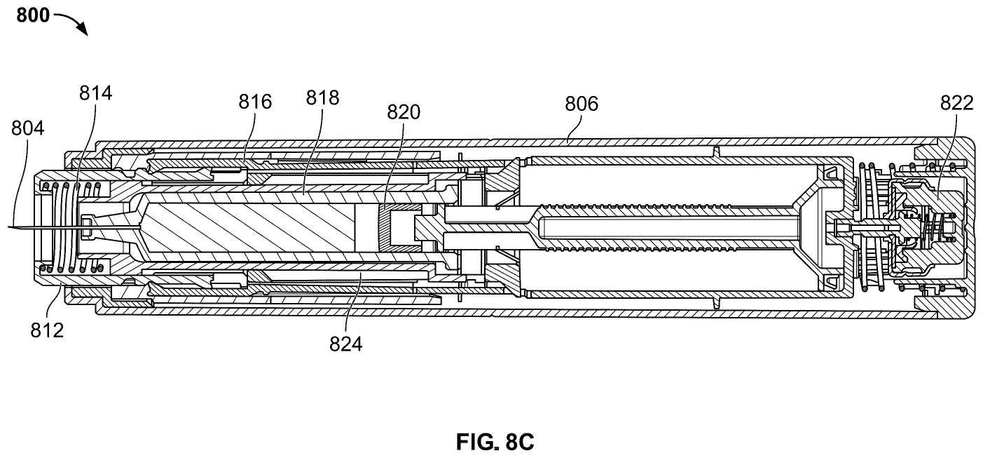

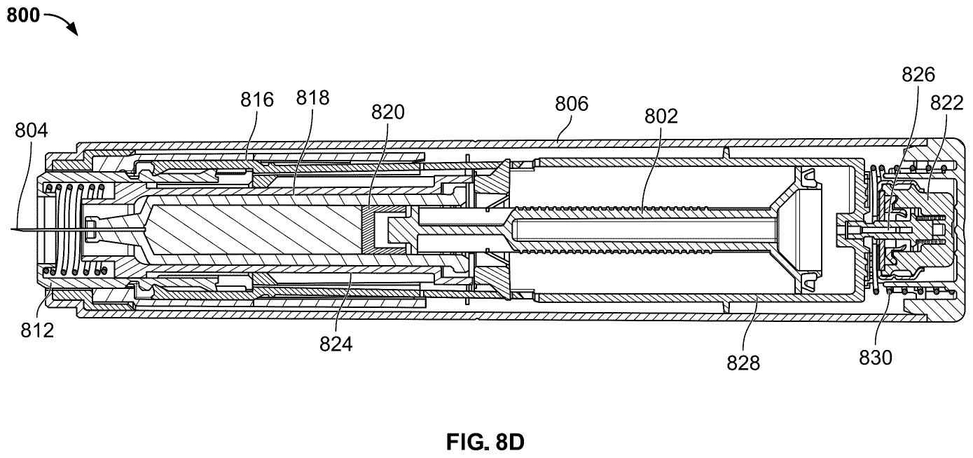

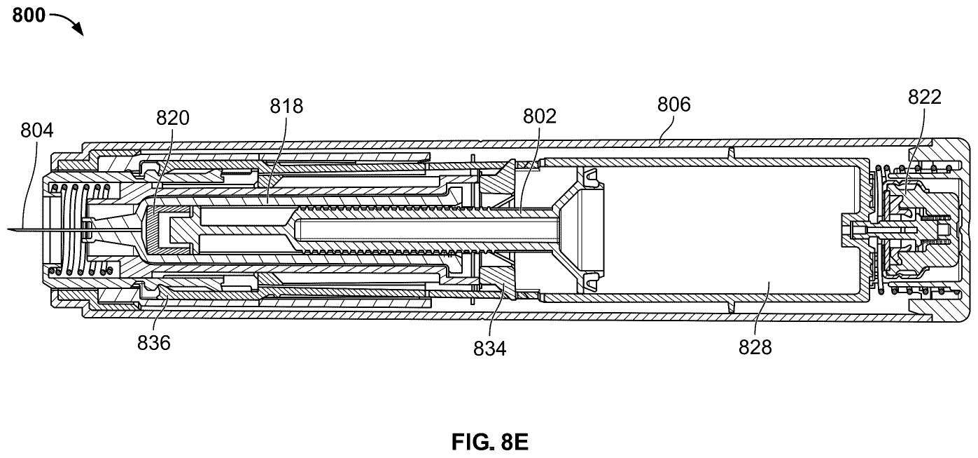

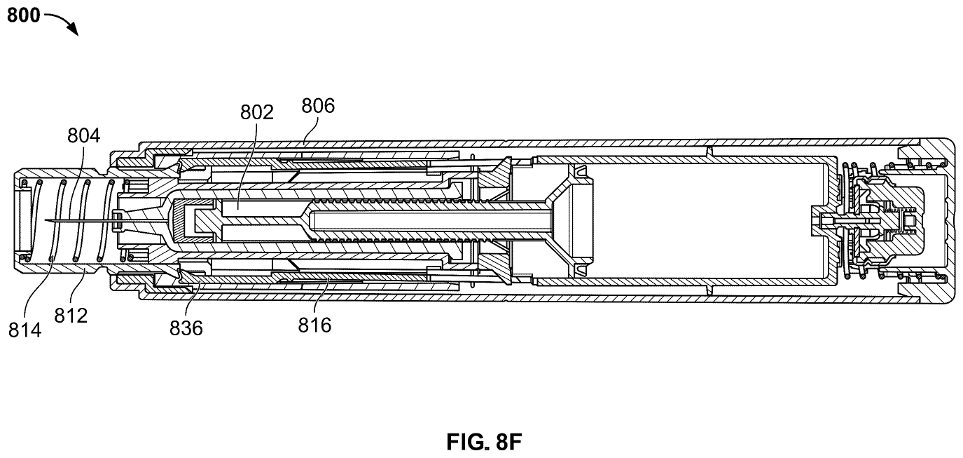

FIG. 6 illustrates the sensing assembly 500 within a proximal portion of an autoinjector 600. The rear cap 502 connects to the housing 602 of the autoinjector 600. A needle guard of the autoinjector 600 is configured to move during injection (see discussion below). When the needle guard moves, a delivery chamber 604 moves in a proximal direction towards the sensing assembly. In turn, movement of the delivery chamber 604 moves a latching can 606 in the proximal direction towards the sensing assembly 500. The latching can 606 contacts the floating probe assembly 506 and causes the floating probe assembly 506 to contact the force sensor of the PCB sub-assembly 504. As discussed above, the floating probe assembly 506 spreads the load between the latching can 606 and the force sensor in the PCB sub-assembly 504 to reduce variability in the force sensor. In some non-limiting embodiments, the delivery chamber 604 constitutes a pressure vessel 828, as provided in FIG. 8D.

In general, the force measured by the force sensor is proportional to the insertion force of the needle into the user. For example, the needle guard moves proximally with a force equal to the insertion force of the needle into the user. The force sensor measures the force applied to the needle guard through the delivery chamber 604, the latching can 606, and the floating probe assembly 506. If the insertion force is greater than a threshold force, then the needle is inserted a sufficient distance into the patient, e.g., to facilitate effective delivery of the medicament.

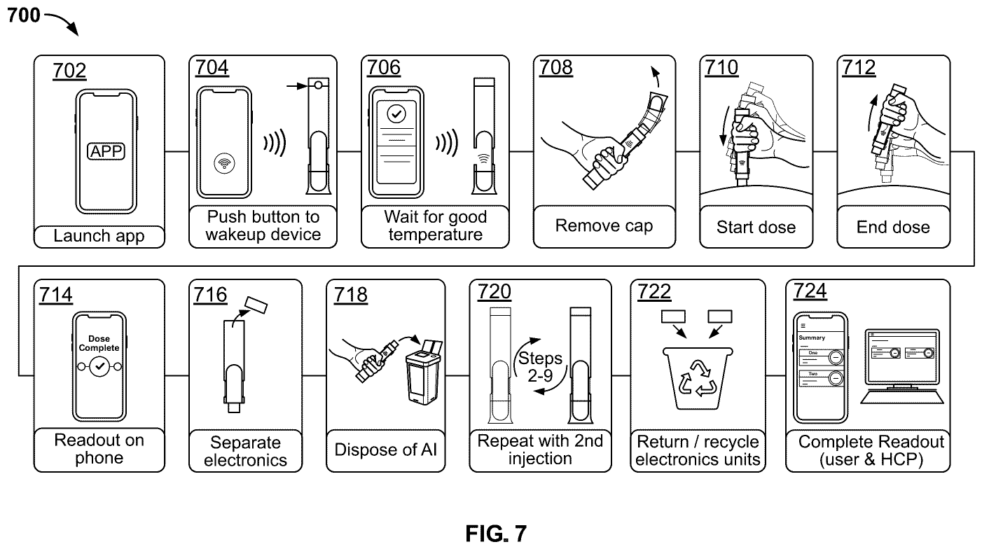

FIG. 7 illustrates an example method 700 of using an autoinjector. The autoinjector can be similar to, e.g., the autoinjector 100 described above. In the method 700, the autoinjector is in communication with a mobile device. For example, the autoinjector can be in communication with the mobile device via a wireless transfer protocol module (see discussion above). In step 702, the method 700 includes launching an app and/or website on the mobile device. In step 704, the method 700 includes pressing a button to wake up the electronics in the autoinjector. For example, such a button can be included on the housing of the autoinjector. The button can be included on the app and/or website on the mobile device. The button may not be activated, but, optionally, activation of the force sensor can wake up the electronics in the autoinjector.

The electronics in the autoinjector can include, e.g., a PCB sub-assembly, a temperature sensor. In step 706, when turned on, the temperature sensor measures a temperature, and the wireless transfer protocol module can communicate the temperature to the mobile device. The temperature sensor can provide any number and type of measurements, including a temperature reading or a rate of change of temperature (e.g., 0.01-0.2° C./minute, 0.01-0.1° C./minute, 0.02-0.2° C./minute, 0.02-0.1° C./minute, about 0.05° C./minute, etc.) determined by readings determined over a time interval (e.g., a time interval of one, two, three, four, or five minutes, in which measurements can be taken every 10, 20, 30, 45, 60, or more seconds). The temperature may be an ambient temperature determined within the autoinjector. While not a direct measurement, the ambient temperature can be used to infer the temperature of the medicament. The temperature may be of a medicament contained within the autoinjector. The user can wait until the medicament is at an appropriate temperature for injection. For example, in many cases, users keep autoinjectors in a refrigerator or other chilled location. After the autoinjector is removed from the refrigerator, the medicament warms up to an appropriate temperature for injection (e.g., 5-10° C., 5-20° C., 5-30° C., 10-20° C., 10-30° C., 20-30° C., less than 30° C., etc.).

When the medicament is at an appropriate temperature, the autoinjector is ready for use. In step 708, the user can remove a cap of the autoinjector. Removing the cap of the autoinjector reveals the needle so that the needle can be inserted into the patient (see discussion below). In step 710, the user inserts the needle into his or her skin to start the dose. A time delay may be present between a time for inserting the needle into the skin and a time for beginning the dose of medicament. The internal components of the autoinjector inject the medicament into the user when the user inserts the needle into his or her skin (see discussion below). In step 712, the dose ends after the medicament has been injected into the user.

In step 714, after the dose ends, information about the dose can be displayed on the mobile device. For example, information can include whether the dose was successfully injected, the time of the delivery, the amount of medicament delivered, a likely error in delivery (e.g., early lift off, shallow delivery, etc.). The information can be determined based on received signals from, e.g., the force sensor, the temperature sensor, the vibration sensor, the mobile device.

Optionally, the autoinjector may be a disposable device. For example, the autoinjector can be configured as a single use device. However, as depicted in step 716, the electronics (e.g., the sensing assembly) can be removed from the autoinjector. For example, referring to FIG. 5, the rear cap 502 can be attached and unattached from the rear cap retainer 508 to connect the PCB sub-assembly 504 and the floating probe assembly 506 to the autoinjector and remove the PCB sub-assembly 504 and the floating probe assembly 506 from the autoinjector.

In step 718, after the electronics are removed from the autoinjector, the user can dispose of the autoinjector. The user may be able to place the electronics onto a second autoinjector. For example, the user can have an autoinjector that has not been used and can place the electronics onto a proximal end of that autoinjector. The electronics (e.g., the sensors) can be aligned properly to function within the second autoinjector. In step 720, the user can then perform an injection with a second autoinjector by performing the portions of the method 700 described above. In some non-limiting examples, the removed electronics can be disposed (e.g., to an appropriate waste stream). In other non-limiting examples, the removed electronics can be returned to the manufacturer for possible reassignment in a controlled manner.

In step 722, after the user is finished using the electronics, the user can dispose of the electronics. For example, a user can be finished using the electronics when the sensors stop working (e.g., due to wear and tear) or when the user no longer needs to take the medicament in the autoinjector (e.g., after a prescription is finished). The user can dispose of the electronics, e.g., by recycling the electronics or returning the electronics to his or her healthcare provider.

In step 724, the mobile device and/or another device, e.g., a device of a healthcare provider, can provide a full readout of information from the one or more injections performed by the user. For example, the full readout of information can include information about each of the injections performed by the user, including whether each dose was successfully injected, the time of the delivery, the amount of medicament delivered, etc.

FIGS. 8A-F illustrate example internal components of an autoinjector 800 that can sense a dispensing movement of a piston 802 within the autoinjector 800 and sense whether a needle 804 is inserted into a patient to a sufficient depth (e.g., similar to the autoinjector 100 of FIGS. 1A-1B). FIGS. 8A-F also illustrate movement of the internal components during firing of the autoinjector.

The autoinjector 800 includes a housing 806 with a removable cap 808 attached to a distal end of the housing 806. The removable cap 808 retains a needle shield 810 that covers the needle 804 and maintains the sterility of the needle 804. A needle guard 812 is telescopically arranged within the housing 806 and can be pressed into the housing 806 to reveal the needle 804 and activate the autoinjector 800 (see discussion below). For example, FIG. 8A illustrates the autoinjector 800 with the removable cap 808 attached to the distal end of the housing 806 and with the needle shield 810 covering the needle 804. FIG. 8B illustrates the autoinjector 800 without the removable cap 808 attached to the distal end of the housing. For example, a user can remove the removable cap 808 from the housing 806 before using the autoinjector 800. When the user removes the removable cap 808 from the housing 806 (as illustrated in FIG. 8B), the needle shield 810 is removed from the needle 804.

The needle guard 812 is biased in the distal direction by a needle guard spring 814. A transfer sleeve 816 is operatively connected to the needle guard 812 such that the transfer sleeve 816 moves with the needle guard 812 when the needle guard 812 moves telescopically. For example, FIG. 8C illustrates a telescopic movement of the needle guard 812 to partially expose the needle 804.

The autoinjector 800 also includes a container 818 retained within the housing 806, a plunger 820 slidably disposed within the container 818, and a gas canister 822. The container 818 is held in a container holder 824 connected to the housing 806. The container 818 can contain a medicament (not shown), and the autoinjector 800 can be activated by a user to dispense the medicament from the container 818. For example, a user can fully depress the needle guard 812 into the housing 806 (e.g., by pressing the needle guard 812 and the autoinjector 800 against his or her skin) to pierce the gas canister 822, releasing compressed gas from within the gas canister 822. The compressed gas can include, for example, argon, carbon dioxide, krypton, xenon, etc.

FIG. 8D illustrates the needle guard 812 in a fully depressed position. When the needle guard 812 is fully depressed into the housing 806, the needle guard 812 moves the transfer sleeve 816 proximally, which causes all of the internal components to move a relatively small proximal distance against the bias of an anti-rattle spring 830 disposed at a proximal end of the autoinjector 800. For example, the relatively small proximal distance can be a distance of 1-10 millimeters. A pressure vessel 828 is moved the relatively small distance and activates a firing pin 826 that pierces an end of the gas canister 822. The pressure vessel 828 is connected to the container holder 824 and retains the piston 802 in alignment with the container 818. The anti-rattle spring 830 biases the pressure vessel 828 (and thus the other internal components) in the distal direction, and the pressure vessel 828 is moved proximally against the bias of the anti-rattle spring 830 when the needle guard 812 is pressed into the housing 806. Also, when the needle guard 812 is fully depressed into the housing 806, the needle guard 812 can cause an internal component (e.g., a latching can) to contact a sensing assembly including a force sensor, such that the force sensor can measure the insertion force of the needle 804 into the user (e.g., as described above). The latching can and sensing assembly can be located in any useful position that does not interfere with actuation of the anti-rattle spring 830 and/or the movement of the pressure vessel 828. The latching may be disposed within an inner volume defined by the anti-rattle spring 830, and the sensing assembly is located in a proximal direction from the latching can and the anti-rattle spring 830.

When the pressure vessel 828 activates the firing pin 826, the compressed gas contained within the gas canister 822 expands into the pressure vessel 828, driving the piston 802 against the plunger 820 and through the container 818. For example, FIGS. 8D and 8E illustrate the piston beginning to move distally to dispense medicament and the end of delivery, respectfully. As the plunger 820 moves distally through the container 818, the medicament is dispensed through the needle 804. The expansion of the compressed gas can provide a force (e.g., 150 Newtons (N), 100 N, 200 N, 100-150 N, 150-200 N, 125-175 N, etc.) to dispense the medicament from the needle 804 within a desired delivery time (e.g., under 50 seconds, under 30 seconds, etc.). Rapidly expanding gas (e.g., released from a gas canister 822) can provide the requisite force, e.g., for subcutaneous injections. For example, a rapidly expanding gas such as described with respect to the autoinjector 800 can provide a larger force than would be provided in an autoinjector that uses a drive spring.

As the piston 802 moves through the dispensing movement, the piston 802 contacts an audible clicker 834 to produce sounds, vibrations, etc. that can be detected (e.g., by a sensing assembly as described above) to track the dispensing movement of the piston 802. For example, the piston 802 can be similar to the plunger rod 202 of FIG. 2, and the audible clicker 834 can be similar to the audible clicker 200 of FIG. 2. For example, the audible clicker 834 can be in the form of a clicker ring having a deflectable protrusion that contacts the piston 802. The dispensing movement of the piston 802 deflects the deflectable protrusion of the audible clicker 834 to cause the audible clicker 834 to produce one or more audible clicks, vibrations, etc. A surface of the piston 802 can be configured to deflect the deflectable protrusion (see discussion above with reference to FIG. 2). Optionally, the audible clicker 834 can be attached to one or more components to prevent movement of the audible clicker 834 as the piston 802 moves through the dispensing movement. The audible clicker 834 may be attached to a lock ring that in turn attaches to the transfer sleeve 816.

As an example, the container 818 may have an internal volume to hold 5 mL of medicament. The container 818 may contain a variety of volumes of medicament (e.g., 1-10 mL of medicament, 2-8 mL of medicament, 4-6 mL of medicament, 3-10 mL of medicament, etc.). The container 818 may have an internal volume of 1.5-3 mL. The container 818 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL.

Optionally, the autoinjector 800 may include a number of vent holes that allow the compressed gas to exit the autoinjector 800 after the dose is completely dispensed. For example, the pressure vessel 828 and the housing 806 can include vent holes. The positions of the vent holes can allow the compressed gas to push the piston 802 through the container 818, and the vent holes do not allow gas to escape during the dispensing. For example, the vent holes are not accessible to the expanding gas until the piston 802 has moved to an end-of-dose distal position. Once the piston 802 reaches the end-of-dose position, the gas can access the vent holes to release the pressure from within the autoinjector 800.

After the piston 802 completes the dispensing movement and the dose is fully dispensed, the user can remove the needle guard 812 from his or her skin. The needle guard spring 814 biases the needle guard 812 in the distal direction, causing the needle guard 812 to extend from the housing 806 and cover the needle 804. For example, FIG. 8F illustrates the needle guard 812 extending from the housing 806 after the dose is dispensed. Covering the needle 804 with the needle guard 812 after the dose is dispensed can be advantageous, e.g., to prevent accidental needle sticks. The transfer sleeve 816 can include at least one locking arm 836 that prevent the needle guard 812 from being pressed into the housing 806 multiple times. Before the needle guard 812 is fully extended (e.g., in FIGS. 8A-E), the locking arm 836 is arranged radially with respect to the needle guard 812. However, when the needle guard 812 is fully extended (e.g., in FIG. 8F), the locking arm 836 can deflect into a locking position proximal to the needle guard 812 to prevent the needle guard 812 from moving proximally.

As used herein, the term “medicament” refers to a pharmaceutical formulation containing at least one active pharmaceutical ingredient (API) which is formulated for administration via injection (e.g., a liquid formulation). The API may be a compound which acts as an anti-viral or has anti-viral properties. The anti-viral API may be a compound that can treat or prevent viral disease such as HIV infection, hepatitis, or other viral disease. The anti-viral API of the medicament may treat or prevent HIV infection alone or in combination with other drugs.

The API may be an anti-HIV drug such as a capsid inhibitor (e.g., an antiretroviral drug that targets the capsid protein shell of viruses). Examples of suitable capsid inhibitors can be found, for example, in U.S. Patent Nos.: 9,951,043; 10,071,985; 11,944,611; 11,787,825; and 12,084,467; and U.S. Publication No.: 20230212148.

The capsid inhibitor may be lenacapavir sodium (SUNLENCA). As used herein, “lenacapavir” (or LEN) refers to N-((S)-1-(3-(4-chloro-3-(methylsulfonamido)-1-(2,2,2-trifluoroethyl)-1H-indazol-7-yl)-6-(3-methyl-3-(methylsulfonyl) but-1-yn-1-yl)pyridin-2-yl)-2-(3,5-difluorophenyl)ethyl)-2-((3bS,4aR)-5,5-difluoro-3-(trifluoromethyl)-3b,4,4a, 5-tetrahydro-1H-cyclopropa[3,4]cyclopenta[1,2-c]pyrazol-1-yl)acetamide.

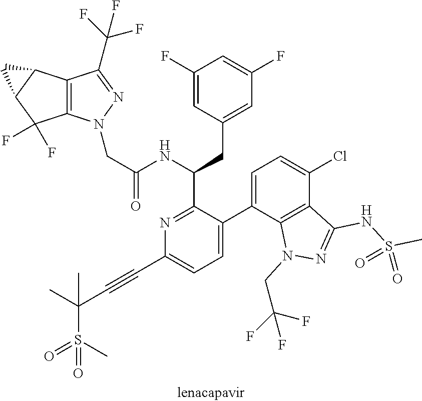 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Synthesis and characterization of lenacapavir, and salts thereof, are described, for example, in US patent publications US20180051005 and US20190300505. Various forms and/or uses of lenacapavir are disclosed, for example, in US 20190083478, US 20190084963, US 20200038389A1, and US 20210188815.

The capsid inhibitor may be lenacapavir, or a pharmaceutically acceptable salt thereof. The capsid inhibitor may be lenacapavir sodium. The capsid inhibitor may be lenacapavir (i.e., the free acid form of lenacapavir).

The present disclosure further includes medicaments (i.e., pharmaceutical compositions) comprising a capsid inhibitor provided herein, or a pharmaceutically acceptable salt thereof, and at least one pharmaceutically acceptable carrier. As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Pharmaceutical compositions of the disclosure are formulated so as to allow the active ingredients contained therein to be bioavailable upon administration of the composition to a patient. Compositions that will be administered to a patient take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the API, or a pharmaceutically acceptable salt thereof (e.g., for treatment or prevention of an HIV infection or reducing the risk of acquiring HIV).

Examples of suitable excipients are well known to the person skilled in the art of parenteral formulations and can be found, for example, in the Handbook of Pharmaceutical Excipients (eds. Rowe, Sheskey & Quinn), 6th edition 2009.

Examples of excipients in a parenteral formulation (for example, a subcutaneous or intramuscular formulation) include polyethylene glycol. In general, polyethylene glycol (PEG) is a polyether having a general formula H—(O—CH₂—CH₂)ₙ—OH. The PEG may be “capped” by an alkyl group. Optionally, the capped PEG may be of the formula alkyl-(O—CH₂—CH₂)ₙ—O-alkyl (for example, CH₃—(O—CH₂—CH₂)ₙ—OCH₃). The pharmaceutical compositions of the present disclosure can include PEG having an average molecular weight of about 100 to about 1000. The average molecular weight of PEG within the pharmaceutical composition may be about 100 to about 800. The average molecular weight of PEG within the pharmaceutical composition may be about 200 to about 600. The average molecular weight of PEG within the pharmaceutical composition may be about 400. The average molecular weight of PEG within the pharmaceutical composition may be about 300. The average molecular weight of PEG within the pharmaceutical composition may be about 200. In some embodiments of the pharmaceutical composition, different molecular weight PEG can be combined to obtain a desired property or properties (for example, viscosity). Specific examples of PEG include, but are not limited to, PEG 100, PEG 200, PEG 300, PEG 400, PEG 500, and PEG 600. PEG 100, for example, refers to a polyethylene glycol with an average molecular weight of about 100.

The pharmaceutical compositions of the present disclosure can be in the form of a sterile injectable preparation, such as a solution or sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution, and isotonic sodium chloride solution. In addition, sterile fixed oils can conventionally be employed as a solvent or suspending medium. For this purpose, any bland fixed oil can be employed, including synthetic mono-or diglycerides. In addition, fatty acids such as oleic acid can likewise be used in the preparation of injectables.

Example formulations suitable for use with the systems and devices of the present application can be found, for example, in U.S. Pat. No.: 11,807,625.

The pharmaceutical composition may comprise the API (e.g., lenacapavir or lenacapavir sodium) at a concentration of about 10 mg/mL to about 600 mg/mL. The pharmaceutical composition may comprise the API (e.g., lenacapavir or lenacapavir sodium) at a concentration of about 100 mg/mL to about 400 mg/mL. The pharmaceutical composition may comprise the API (e.g., lenacapavir or lenacapavir sodium) at a concentration of about 250 mg/mL to about 350 mg/mL. The pharmaceutical composition may comprise the API (e.g., lenacapavir or lenacapavir sodium) at a concentration of about 300 mg/mL to about 325 mg/mL. The pharmaceutical composition may comprise the API (e.g., lenacapavir or lenacapavir sodium) at a concentration of about 305 mg/mL to about 310 mg/mL. The pharmaceutical composition may comprise the API (e.g., lenacapavir or lenacapavir sodium) at a concentration of about 309 mg/mL.

The solution provided herein may comprise lenacapavir, or a pharmaceutically acceptable salt thereof, PEG 300, and water. The solution may comprise lenacapavir sodium, PEG 300, and water. The solution may comprise lenacapavir, PEG 300, and water.

The amount of water in the solution comprising lenacapavir sodium, PEG 300, and water may be about 10 w/w % to about 40 w/w %. The amount of water in the solution comprising lenacapavir sodium, PEG 300, and water may be about 15 w/w % to about 35 w/w %. The amount of water in the solution comprising lenacapavir sodium, PEG 300, and water may be about 20 w/w % to about 30 w/w %. The amount of water in the solution comprising lenacapavir sodium, PEG 300, and water may be about 21 w/w % to about 29 w/w %.

The amount of water in the solution comprising lenacapavir sodium, PEG 300, and water may be about 21.1 w/w % to about 27.5 w/w %. The amount of water in the solution comprising lenacapavir sodium, PEG 300, and water may be about 21.13 w/w % to about 27.47 w/w %. The amount of water in the solution comprising lenacapavir sodium, PEG 300, and water may be about 21.1 w/w %. The amount of water in the solution comprising lenacapavir sodium, PEG 300, and water may be about 21.13 w/w %. The amount of water in the solution comprising lenacapavir sodium, PEG 300, and water may be about 23 w/w %. The amount of water in the solution comprising lenacapavir sodium, PEG 300, and water may be about 23.4 w/w %. The amount of water in the solution comprising lenacapavir sodium, PEG 300, and water may be about 23.41 w/w %.

The amount of PEG 300 in the solution comprising lenacapavir sodium, PEG 300, and water may be about 35 w/w % to about 75 w/w %. The amount of PEG 300 in the solution comprising lenacapavir sodium, PEG 300, and water may be about 40 w/w % to about 55 w/w %. The amount of PEG 300 in the solution comprising lenacapavir sodium, PEG 300, and water may be about 43 w/w % to about 47 w/w %. The amount of PEG 300 in the solution comprising lenacapavir sodium, PEG 300, and water may be about 45 w/w %. The amount of PEG 300 in the solution comprising lenacapavir sodium, PEG 300, and water may be about 45.25 w/w %. The amount of PEG 300 in the solution comprising lenacapavir sodium, PEG 300, and water may be about 48 w/w % to about 52 w/w %. The amount of PEG 300 in the solution comprising lenacapavir sodium, PEG 300, and water may be about 50 w/w %. The amount of PEG 300 in the solution comprising lenacapavir sodium, PEG 300, and water may be about 50.1 w/w %. The amount of PEG 300 in the solution comprising lenacapavir sodium, PEG 300, and water may be about 50.13 w/w %.

The amount of lenacapavir sodium in the solution comprising PEG 300 and water may be about 5 w/w % to about 35 w/w %. The amount of lenacapavir sodium in the solution comprising PEG 300 and water may be about 20 w/w % to about 35 w/w %.

The amount of lenacapavir sodium in the solution comprising PEG 300 and water may be about 20 w/w % to about 30 w/w %. The amount of lenacapavir sodium in the solution comprising PEG 300 and water may be about 24 w/w % to about 28 w/w %. The amount of lenacapavir sodium in the solution comprising PEG 300 and water may be about 26.5 w/w %. The amount of lenacapavir sodium in the solution comprising PEG 300 and water may be about 26.46 w/w %.

The amount of lenacapavir sodium in the solution comprising PEG 300 and water may be about 30 w/w % to about 35 w/w %. The amount of lenacapavir sodium in the solution comprising PEG 300 and water may be about 32 w/w % to about 34 w/w %. The amount of lenacapavir sodium in the solution comprising PEG 300 and water may be about 33.6 w/w %. The amount of lenacapavir sodium in the solution comprising PEG 300 and water may be about 33.61 w/w %.

The solution may comprise about 10 w/w % to about 40 w/w % water, about 35 w/w % to about 75 w/w % PEG 300, and about 5 w/w % to about 45 w/w % of lenacapavir sodium. The solution may comprise about 10 w/w % to about 30 w/w % water, about 35 w/w % to about 65 w/w % PEG 300, and about 5 w/w % to about 45 w/w % of lenacapavir sodium.

The solution may comprise about 21.13 w/w % to about 27.47 w/w % water, about 45.25 w/w % to about 58.84 w/w % PEG 300, and about 13.69 w/w % to about 33.61 w/w % of lenacapavir sodium. The solution may comprise about 21.1 w/w % water, about 45.3 w/w % PEG 300, and about 33.6 w/w % of lenacapavir sodium. The solution may comprise about 21.13 w/w % water, about 45.25 w/w % PEG 300, and about 33.61 w/w % of lenacapavir sodium. The solution may comprise about 23.4 w/w % water, about 50.1 w/w % PEG 300, and about 26.5 w/w % of lenacapavir sodium. The solution may comprise about 23.41 w/w % water, about 50.13 w/w % PEG 300, and about 26.46 w/w % of lenacapavir sodium.

The solution provided herein may further comprise ethanol. The solution may comprise about 10 w/w % to about 40 w/w % water, about 20 w/w % to about 75 w/w % PEG 300, about 10 w/w % to about 70 w/w % of lenacapavir sodium, and about 1 w/w % to about 10 w/w % of ethanol. The solution may comprise about 10 w/w % to about 20 w/w % water, about 30 w/w % to about 40 w/w % PEG 300, about 37 w/w % to about 45 w/w % of lenacapavir sodium, and about 3 w/w % to about 8 w/w % of ethanol.

The solution may comprise about 16.93 w/w % water, about 36.22 w/w % PEG 300, about 41.85 w/w % of lenacapavir sodium, and about 5.00 w/w % ethanol. The solution may comprise about 16.9 w/w % water, about 36.2 w/w % PEG 300, about 41.9 w/w % of lenacapavir sodium, and about 5.0 w/w % ethanol.

The medicament which is administered by the injection device of the invention may be a long-acting injectable formulation which can be administered to a patient, for example, twice per month, once per month, once per quarter (e.g., every 3 months), twice per year (e.g., every 6 months), once per year, or less frequently.

Additionally, the medicament can include a formulation which has relatively high viscosity. The medicament may have a viscosity of 100-1000 cP. Preferably, the medicament may have a viscosity of 100-600 cP. For example, the medicament may have a viscosity of 250-500 cP, 150-350 cP or 450-600 cP. The medicament may be a solution comprising lenacapavir sodium the medicament may have a viscosity of 250-500 cP, 150-350 cP or 450-600 cP.

A number of embodiments have been described. Nevertheless, it will be understood that various modifications may be made without departing from the spirit and scope of the disclosure. Accordingly, other embodiments are within the scope of the following claims.

### Embodiments

The following list provides embodiments of the invention and forms part of the description. These embodiments can be combined in any compatible combination beyond those expressly stated. The embodiments can also be combined with any compatible features described herein:

1. An autoinjector comprising:

a housing;

a container disposed within the housing and configured to contain medicament;

a plunger slidably disposed within the container;

a plunger rod configured to push the plunger through the container to dispense the medicament when the container contains medicament;

an audible clicker contacting the plunger rod, wherein a dispensing movement of the plunger rod deflects the audible clicker causing the audible clicker to produce one or more audible clicks; and

a sensor configured to detect the one or more audible clicks of the audible clicker as the plunger rod moves.

2. An autoinjector, comprising:

a housing;

a container disposed within the housing and configured to contain medicament;

a plunger slidably disposed within the container;

a plunger rod configured to push the plunger through the container to dispense the medicament when the container contains medicament;

a mechanism configured so that a dispensing movement of the plunger rod causes the mechanism to generate a sound; and

a sensor configured to detect sound generated by the mechanism.

3. The autoinjector of embodiment 1 or embodiment 2, wherein the one or more audible clicks or the sound are configured to indicate dose progression.

4. The autoinjector of embodiment 1 or embodiment 2, wherein the one or more audible clicks or the sound are configured to be detected for tracking the dispensing movement.

5. The autoinjector of any of embodiments 1-4, wherein the one or more audible clicks or the sound are configured to indicate completion of the dispensing movement of the plunger rod.

6. The autoinjector of any of embodiments 1-5, wherein the container contains the medicament.

7. The autoinjector of any preceding embodiment, wherein the medicament comprises lenacapavir or a pharmaceutically accepted salt thereof.

8. The autoinjector of embodiment 7, wherein the medicament comprises lenacapavir sodium.

9. The autoinjector of embodiment 8, wherein the medicament is a solution comprising: lenacapavir sodium, PEG 300 and water.

10. The autoinjector of embodiment 9, wherein the solution comprises about 21.13 w/w % water, about 45.25 w/w % PEG 300, and about 33.61 w/w % of lenacapavir sodium.

11. The autoinjector of embodiment 9, wherein the solution comprises about 23.41 w/w % water, about 50.13 w/w % PEG 300, and about 26.46 w/w % of lenacapavir sodium.

12. The autoinjector of embodiment 9, wherein the solution further comprises ethanol, optionally wherein the solution comprises about 16.9 w/w % water, about 36.2 w/w % PEG 300, about 41.9 w/w % of lenacapavir sodium, and about 5.0 w/w % ethanol.

13. The autoinjector of any preceding embodiment, wherein the autoinjector further comprises a gas canister assembly configured to release pressurized gas which, when released, provides a force acting on the plunger rod to push the plunger through the container.

14. The autoinjector of any of embodiments 1-13, wherein the sensor is disposed in a proximal end of the housing.

15. The autoinjector of any of embodiments 1-14 when dependent upon embodiment 1, wherein the audible clicker is configured to produce a plurality of audible clicks during the dispensing movement of the plunger rod.

16. The autoinjector of any of embodiments 1-15 when dependent upon embodiment 1, wherein the audible clicker is configured to produce 1-100, 1-50, 1-20, 2-100, 2-50, 2-20, 5-100, 5-50, 5-20, or 5-15 audible clicks during the dispensing movement of the plunger rod.

17. The autoinjector of any of embodiments 1-16 when dependent upon embodiment 1, wherein the audible clicker comprises a ring surrounding the plunger rod, the ring comprising a deflectable protrusion that is configured to contact the plunger rod and produces the one or more audible clicks.

18. The autoinjector of embodiment 17, wherein the plunger comprises a ridged surface that is configured to contact the deflectable protrusion of the audible clicker.

19. The autoinjector of embodiment 18, wherein each ridge of the ridged surface is configured to cause the deflectable protrusion to deflect during the dispensing movement of the plunger rod.

20. The autoinjector of embodiment 18 or embodiment 19, wherein a first ridge of the plunger is spaced such that the first ridge is configured to contact the audible clicker when the plunger rod contacts the plunger or before the plunger rod contacts the plunger.

21. A system comprising:

an autoinjector according to any one of embodiments 1-15; and

a processor configured to process data generated by the sensor to track the dispensing movement of the plunger rod.

22. A method of detecting a dispensing movement of a plunger rod within an autoinjector, the method comprising:

detecting, using a sensor, one or more audible clicks of an audible clicker as the plunger rod moves during the dispensing movement, wherein the dispensing movement of the plunger rod deflects the audible clicker causing the audible clicker to produce the one or more audible clicks.

23. A method of detecting a dispensing movement of a plunger rod within an autoinjector, the method comprising:

detecting a sound generated due to movement of the plunger rod during the dispensing movement.

24. The method of embodiment 22 or embodiment 23, wherein the sensor is disposed in a proximal end of the autoinjector.

25. The method of embodiment 22, wherein the audible clicker is configured to produce a plurality of audible clicks during the dispensing movement of the plunger rod.

26. The method of embodiment 22 or embodiment 25, wherein the audible clicker is configured to produce 1-100, 1-50, 1-20, 2-100, 2-50, 2-20, 5-100, 5-50, 5-20, or 5-15 audible clicks during the dispensing movement of the plunger rod.

27. The method of embodiment 22, embodiment 25 or embodiment 26, wherein the audible clicker comprises a ring surrounding the plunger rod, the ring comprising a deflectable protrusion that contacts the plunger rod and produces the one or more audible clicks.

28. The method of embodiment 27, wherein the plunger comprises a ridged surface that contacts the deflectable protrusion of the audible clicker.

29. The method of embodiment 28, wherein each ridge of the ridged surface causes the deflectable protrusion to deflect during the dispensing movement of the plunger rod.

30. The method of embodiment 28 or embodiment 29, wherein a first ridge of the plunger is spaced such that the first ridge contacts the audible clicker when the plunger rod contacts the plunger or before the plunger rod contacts the plunger.

31. The method of any one of embodiments 22-30, further comprising sending, via a wireless transfer protocol module, signals from the sensor to a mobile device.

32. The method of embodiment 31, further comprising displaying information about the dispensing movement on the mobile device.

33. The method of any one of embodiments 22-32, further comprising measuring a temperature of medicament within the autoinjector or an ambient temperature within the autoinjector using a temperature sensor.

34. The method of embodiment 33, further comprising sending, via a wireless transfer protocol module, signals from the temperature sensor to a mobile device.

35. The method of embodiment 34, further comprising displaying the temperature of the medicament or the ambient temperature on the mobile device.

36. An autoinjector comprising:

a housing;

a container disposed within the housing and configured to contain medicament;

a plunger slidably disposed within the container;

a plunger rod configured to push the plunger through the container to dispense the medicament when the container contains medicament;

a deflectable protrusion contacting the plunger rod, wherein a dispensing movement of the plunger rod deflects the deflectable protrusion; and

a sensor configured to detect one or more deflections of the deflectable protrusion as the plunger rod moves.

37. An autoinjector, comprising:

a housing;

a container disposed within the housing and configured to contain medicament;

a plunger slidably disposed within the container;

a plunger rod configured to push the plunger through the container to dispense the medicament when the container contains medicament;

a mechanism configured so that a dispensing movement of the plunger rod causes the mechanism to vibrate; and

a sensor configured to detect vibration generated by the mechanism.

38. The autoinjector of embodiment 36 or embodiment 37, wherein the one or more deflections or the vibration are configured to indicate dose progression.

39. The autoinjector of embodiment 36 or embodiment 37, wherein the one or more deflections or the vibration are configured to be detected for tracking the dispensing movement.

40. The autoinjector of any of embodiments 36-39, wherein the one or more deflections or the vibration are configured to indicate completion of the dispensing movement of the plunger rod.

41. The autoinjector of any of embodiments 36-40, wherein the container contains the medicament.

42. The autoinjector of any of embodiments 36-41, wherein the medicament comprises lenacapavir or a pharmaceutically accepted salt thereof.

43. The autoinjector of embodiment 42, wherein the medicament comprises lenacapavir sodium.

44. The autoinjector of embodiment 43, wherein the medicament is a solution comprising: lenacapavir sodium, PEG 300 and water.

45. The autoinjector of embodiment 44, wherein the solution comprises about 21.13 w/w % water, about 45.25 w/w % PEG 300, and about 33.61 w/w % of lenacapavir sodium.

46. The autoinjector of embodiment 44, wherein the solution comprises about 23.41 w/w % water, about 50.13 w/w % PEG 300, and about 26.46 w/w % of lenacapavir sodium.

47. The autoinjector of embodiment 44, wherein the solution further comprises ethanol, optionally wherein the solution comprises about 16.9 w/w % water, about 36.2 w/w % PEG 300, about 41.9 w/w % of lenacapavir sodium, and about 5.0 w/w % ethanol.

48. The autoinjector of any of embodiments 36-47, wherein the autoinjector further comprises a gas canister assembly configured to release pressurized gas which, when released, provides a force acting on the plunger rod to push the plunger through the container.

49. The autoinjector of any of embodiments 36-48, wherein the sensor is disposed in a proximal end of the housing.

50. The autoinjector of embodiment 36, wherein the dispensing movement of the plunger rod causes the deflectable protrusion to deflect a plurality of times.

51. The autoinjector of embodiment 36 or embodiment 50, wherein the dispensing movement of the plunger rod causes the deflectable protrusion to deflect 1-100, 1-50, 1-20, 2-100, 2-50, 2-20, 5-100, 5-50, 5-20, or 5-15 times.

52. The autoinjector of embodiment 36, embodiment 50 or embodiment 51, wherein the autoinjector comprises a ring surrounding the plunger rod, and the ring comprises the deflectable protrusion.

53. The autoinjector of embodiment 52, wherein the plunger comprises a ridged surface that is configured to contact the deflectable protrusion.

54. The autoinjector of embodiment 53, wherein each ridge of the ridged surface is configured to cause the deflectable protrusion to deflect during the dispensing movement of the plunger rod.

55. The autoinjector of embodiment 53 or embodiment 54, wherein a first ridge of the plunger is spaced such that the first ridge is configured to contact the deflectable protrusion when the plunger rod contacts the plunger or before the plunger rod contacts the plunger.

56. The autoinjector of any one of embodiments 36-55, wherein the sensor comprises an accelerometer.

57. The autoinjector of any one of embodiments 36-55, wherein the sensor comprises a displacement sensor.

58. The autoinjector of any one of embodiments 36-55, wherein the sensor comprises a velocity sensor.

59. A system, comprising:

an autoinjector according to any one of embodiments 36-58; and

a processor configured to process data generated by the sensor to track the dispensing movement of the plunger rod.

60. The system of embodiment 59, wherein the sensor is disposed in a proximal end of the housing.

61. The system of embodiment 59, wherein the processor is within the housing.

62. The system of embodiment 59, wherein the processor is external to the housing.

63. A method of detecting a dispensing movement of a plunger rod within an autoinjector comprising a plunger rod and a protrusion contacting the plunger rod, the method comprising:

detecting, using a sensor, one or more deflections of the protrusion as the plunger rod moves during the dispensing movement, wherein the dispensing movement of the plunger rod deflects the protrusion.

64. A method of detecting a dispensing movement of a plunger rod within an autoinjector comprising a component, the method comprising:

detecting a deflection of the component due to movement of the plunger rod during the dispensing movement.

65. The method of embodiment 63 or embodiment 64, wherein the sensor is disposed in a proximal end of the autoinjector.

66. The method of embodiment 63, wherein the dispensing movement of the plunger rod causes the deflectable protrusion to deflect a plurality of times.

67. The method of embodiment 63 or embodiment 66, wherein the dispensing movement of the plunger rod causes the deflectable protrusion to deflect 1-100, 1-50, 1-20, 2-100, 2-50, 2-20, 5-100, 5-50, 5-20, or 5-15 times.

68. The method of embodiment 63, embodiment 66 or embodiment 67, the plunger comprises a ridged surface that is configured to contact the deflectable protrusion.

69. The method of embodiment 68, wherein each ridge of the ridged surface is configured to cause the deflectable protrusion to deflect during the dispensing movement of the plunger rod.

70. The method of embodiment 69, wherein each ridge of the ridged surface causes the deflectable protrusion to deflect during the dispensing movement of the plunger rod.

71. The method of embodiment 68 or embodiment 69, wherein a first ridge of the plunger is spaced such that the first ridge contacts the audible clicker when the plunger rod contacts the plunger or before the plunger rod contacts the plunger.

72. The method of any one of embodiments 63-71 further comprising sending, via a wireless transfer protocol module, signals from the sensor to a mobile device.

73. The method of embodiment 72, further comprising displaying information about the dispensing movement on the mobile device.

74. The method of any one of embodiments 63-73, further comprising measuring a temperature of medicament within the autoinjector or an ambient temperature within the autoinjector using a temperature sensor.

75. The method of embodiment 74, further comprising sending, via a wireless transfer protocol module, signals from the temperature sensor to a mobile device.

76. The method of embodiment 75, further comprising displaying the temperature of the medicament or the ambient temperature on the mobile device.

77. An autoinjector comprising:

a housing;

a needle arranged at a distal end of the housing;

a force sensor configured to detect that an insertion force is greater than a threshold force; and

a processor configured to use the detected insertion force to determine when the insertion force is greater than a threshold insertion force, and thus determine that an insertion distance of the needle into a user is greater than a threshold insertion distance.

78. The autoinjector of embodiment 77, wherein the processor is at least partially disposed within the housing.

79. The autoinjector of embodiment 77, wherein the processor is completely external to the housing.

80. The autoinjector of any of embodiments 77-79, wherein the force sensor is disposed in a proximal end of the housing.

81. The autoinjector of any one of embodiments 77-80, wherein, during an insertion of the needle into the user, at least one internal component of the autoinjector is configured to contact the force sensor with a force proportional to the insertion force.

82. The autoinjector of embodiment 81, wherein the at least one internal component comprises a needle guard telescopically arranged within the housing.

83. The autoinjector of embodiment 81, wherein the at least one internal component comprises a container disposed within the housing and configured to contain medicament, a plunger slidably disposed within the container, and a plunger rod configured to push the plunger through the container to dispense the medicament when the container contains medicament.

84. The autoinjector of any one of embodiments 77-83, wherein the force sensor comprises a load cell.

85. The autoinjector of any one of embodiments 77-83, wherein the force sensor comprises a strain gauge.

86. The autoinjector of any one of embodiments 77-83, wherein the force sensor comprises a force sensitive resistor.

87. The autoinjector of any one of embodiments 77-83, wherein the force sensor comprises a force sensitive capacitor.

88. A system, comprising:

an autoinjector comprising:

a housing;
a needle arranged at a distal end of the housing; and
a force sensor configured to detect that an insertion force is greater than a threshold force; and

a processor external to the autoinjector,

wherein the processor is configured to use the detected insertion force to determine when the insertion force is greater than a threshold insertion force, and thus determine that an insertion distance of the needle into a user is greater than a threshold insertion distance.

89. The system of embodiment 88, wherein, during an insertion of the needle into the user, at least one internal component of the autoinjector is configured to contact the force sensor with a force proportional to the insertion force.

90. The system of embodiment 89, wherein the at least one internal component comprises a needle guard telescopically arranged within the housing.

91. The system of embodiment 89, wherein the at least one internal component comprises a container disposed within the housing and configured to contain medicament, a plunger slidably disposed within the container, and a plunger rod configured to push the plunger through the container to dispense the medicament when the container contains medicament.

92. The system of any one of embodiments 88-91, wherein the force sensor comprises a load cell.

93. The system of any one of embodiments 88-91, wherein the force sensor comprises a strain gauge.

94. The system of any one of embodiments 88-91, wherein the force sensor comprises a force sensitive resistor.

95. The system of any one of embodiments 88-91, wherein the force sensor comprises a force sensitive capacitor.

96. The autoinjector of embodiment 83 or the system of embodiment 91, wherein the container contains the medicament.

97. The autoinjector or system of embodiment 83, embodiment 91 or embodiment 96, wherein the medicament comprises lenacapavir or a pharmaceutically accepted salt thereof.

98. The autoinjector or system of embodiment 97, wherein the medicament comprises lenacapavir sodium.

99. The autoinjector or system of embodiment 98, wherein the medicament is a solution comprising: lenacapavir sodium, PEG 300 and water.

100. The autoinjector or system of embodiment 99, wherein the solution comprises about 21.13 w/w % water, about 45.25 w/w % PEG 300, and about 33.61 w/w % of lenacapavir sodium.

101. The autoinjector or system of embodiment 99, wherein the solution comprises about 23.41 w/w % water, about 50.13 w/w % PEG 300, and about 26.46 w/w % of lenacapavir sodium.

102. The autoinjector or system of embodiment 99, wherein the solution further comprises ethanol, optionally wherein the solution comprises about 16.9 w/w % water, about 36.2 w/w % PEG 300, about 41.9 w/w % of lenacapavir sodium, and about 5.0 w/w % ethanol.

103. The autoinjector or system of any of embodiments 83, 91, 96 or 97, wherein the autoinjector further comprises a gas canister assembly configured to release pressurized gas which, when released, provides a force acting on the plunger rod to push the plunger through the container.

104. A composition comprising lenacapavir or a pharmaceutically accepted salt thereof for use in the prevention or treatment of HIV, wherein the composition is administered via the autoinjector of any of embodiments 1 to 21, 36 to 62, or 77 to 103.

105. The composition of embodiment 104, wherein the composition is subcutaneously administered via the autoinjector, optionally wherein the medicament is a solution comprising either:
(a) about 21.13 w/w % water, about 45.25 w/w % PEG 300, and about 33.61 w/w % of lenacapavir sodium; or
(b) about 23.41 w/w % water, about 50.13 w/w % PEG 300, and about 26.46 w/w % of lenacapavir sodium.

106. The composition of embodiment 104, wherein the composition is intramuscularly administered via the autoinjector, optionally wherein the composition is a solution comprising about 16.9 w/w % water, about 36.2 w/w % PEG 300, about 41.9 w/w % of lenacapavir sodium, and about 5.0 w/w % ethanol.

107. Use of lenacapavir or a pharmaceutically accepted salt thereof for the manufacture of a medicament for the prevention or treatment of HIV, wherein the prevention or treatment comprises administering the medicament via the autoinjector of any of embodiments 1 to 21, 36 to 62, or 77 to 103.

108. The use of embodiment 107, wherein the medicament is subcutaneously administered via the autoinjector, optionally wherein the medicament is a solution comprising either:
(a) about 21.13 w/w % water, about 45.25 w/w % PEG 300, and about 33.61 w/w % of lenacapavir sodium; or
(b) about 23.41 w/w % water, about 50.13 w/w % PEG 300, and about 26.46 w/w % of lenacapavir sodium.

109. The use of embodiment 107, wherein the medicament is intramuscularly administered via the autoinjector, optionally wherein the medicament is a solution comprising about 16.9 w/w % water, about 36.2 w/w % PEG 300, about 41.9 w/w % of lenacapavir sodium, and about 5.0 w/w % ethanol.

1. An autoinjector comprising:
a housing;
a container disposed within the housing and configured to contain medicament;
a plunger slidably disposed within the container;
a plunger rod configured to push the plunger through the container to dispense the medicament when the container contains medicament;
an audible clicker contacting the plunger rod, wherein a dispensing movement of the plunger rod deflects the audible clicker causing the audible clicker to produce one or more audible clicks; and
a sensor configured to detect the one or more audible clicks of the audible clicker as the plunger rod moves.

2. An autoinjector, comprising:
a housing;
a container disposed within the housing and configured to contain medicament;
a plunger slidably disposed within the container;
a plunger rod configured to push the plunger through the container to dispense the medicament when the container contains medicament;
a mechanism configured so that a dispensing movement of the plunger rod causes the mechanism to generate a sound; and
a sensor configured to detect sound generated by the mechanism.

3. The autoinjector of claim 1, wherein the one or more audible clicks or the sound are configured to indicate dose progression.

4. The autoinjector of claim 1, wherein the one or more audible clicks or the sound are configured to be detected for tracking the dispensing movement.

5. The autoinjector of claim 1, wherein the one or more audible clicks are configured to indicate completion of the dispensing movement of the plunger rod.

6. The autoinjector of claims 1, wherein the container contains the medicament.

7. The autoinjector of claim 1, wherein the medicament comprises lenacapavir or a pharmaceutically accepted salt thereof.

8. The autoinjector claim 1, wherein the autoinjector further comprises a gas canister assembly configured to release pressurized gas which, when released, provides a force acting on the plunger rod to push the plunger through the container.

9. The autoinjector of claims 1, wherein the sensor is disposed in a proximal end of the housing.

10. The autoinjector of claim 1, wherein the audible clicker is configured to produce a plurality of audible clicks during the dispensing movement of the plunger rod.

11. The autoinjector of claim 1, wherein the audible clicker is configured to produce 1-100, 1-50, 1-20, 2-100, 2-50, 2-20, 5-100, 5-50, 5-20, or 5-15 audible clicks during the dispensing movement of the plunger rod.

12. The autoinjector of claim 1, wherein the audible clicker comprises a ring surrounding the plunger rod, the ring comprising a deflectable protrusion that is configured to contact the plunger rod and produces the one or more audible clicks.

13. The autoinjector of claim 12, wherein the plunger comprises a ridged surface that is configured to contact the deflectable protrusion of the audible clicker.

14. The autoinjector of claim 13, wherein each ridge of the ridged surface is configured to cause the deflectable protrusion to deflect during the dispensing movement of the plunger rod.

15. The autoinjector of claim 13, wherein a first ridge of the plunger is spaced such that the first ridge is configured to contact the audible clicker when the plunger rod contacts the plunger or before the plunger rod contacts the plunger.

16. A system comprising:
an autoinjector according to claim 1; and
a processor configured to process data generated by the sensor to track the dispensing movement of the plunger rod.

17. (canceled)

18. A method of detecting a dispensing movement of a plunger rod within an autoinjector, the method comprising:
detecting, using a sensor, one or more audible clicks of an audible clicker as the plunger rod moves during the dispensing movement, wherein the dispensing movement of the plunger rod deflects the audible clicker causing the audible clicker to produce the one or more audible clicks.

19. (canceled)

20. The method of claim 18, wherein the sensor is disposed in a proximal end of the autoinjector.

21. The method of claim 18, wherein the audible clicker is configured to produce a plurality of audible clicks during the dispensing movement of the plunger rod.

22. The method of claim 18, wherein the audible clicker is configured to produce 1-100, 1-50, 1-20, 2-100, 2-50, 2-20, 5-100, 5-50, 5-20, or 5-15 audible clicks during the dispensing movement of the plunger rod.

23. The method of claim 18, wherein the audible clicker comprises a ring surrounding the plunger rod, the ring comprising a deflectable protrusion that contacts the plunger rod and produces the one or more audible clicks.

24. The method of claim 23, wherein the plunger comprises a ridged surface that contacts the deflectable protrusion of the audible clicker.

25. The method of claim 24, wherein each ridge of the ridged surface causes the deflectable protrusion to deflect during the dispensing movement of the plunger rod.

26. The method of claim 24, wherein a first ridge of the plunger is spaced such that the first ridge contacts the audible clicker when the plunger rod contacts the plunger or before the plunger rod contacts the plunger.

27. The method of claim 18, further comprising sending, via a wireless transfer protocol module, signals from the sensor to a mobile device.

28. The method of claim 27, further comprising displaying information about the dispensing movement on the mobile device.

29. The method of claim 18, further comprising measuring a temperature of medicament within the autoinjector or an ambient temperature within the autoinjector using a temperature sensor.

30. The method of claim 29, further comprising sending, via a wireless transfer protocol module, signals from the temperature sensor to a mobile device.

31. The method of claim 30, further comprising displaying the temperature of the medicament or the ambient temperature on the mobile device.

32. An autoinjector comprising:
a housing;
a container disposed within the housing and configured to contain medicament;
a plunger slidably disposed within the container;
a plunger rod configured to push the plunger through the container to dispense the medicament when the container contains medicament;
a deflectable protrusion contacting the plunger rod, wherein a dispensing movement of the plunger rod deflects the deflectable protrusion; and
a sensor configured to detect one or more deflections of the deflectable protrusion as the plunger rod moves.

33. An autoinjector, comprising:
a housing;
a container disposed within the housing and configured to contain medicament;
a plunger slidably disposed within the container;
a plunger rod configured to push the plunger through the container to dispense the medicament when the container contains medicament;
a mechanism configured so that a dispensing movement of the plunger rod causes the mechanism to vibrate; and
a sensor configured to detect vibration generated by the mechanism.

34-67. (canceled)

68. An autoinjector comprising:
a housing;
a needle arranged at a distal end of the housing;
a force sensor configured to detect that an insertion force is greater than a threshold force; and
a processor configured to use the detected insertion force to determine when the insertion force is greater than a threshold insertion force, and thus determine that an insertion distance of the needle into a user is greater than a threshold insertion distance.

69-89. (canceled)

90. A composition comprising lenacapavir or a pharmaceutically accepted salt thereof for use in the prevention or treatment of HIV, wherein the composition is administered via the autoinjector of claim 1.

91. Use of lenacapavir or a pharmaceutically accepted salt thereof for the manufacture of a medicament for the prevention or treatment of HIV, wherein the prevention or treatment comprises administering the medicament via the autoinjector of claim 1.
